SEQUENCE LISTING (60 sequences)

SEQ ID NO: 1 - DNA, 817 bp, Homo sapiens
  CDS (106)..(627)
agtcctgcgtccgggccccgaggcgcagcagggcaccaggtggagcaccagctacgcgtggcgcagcgcagcgtccctagcaccgagcctcccgcagccgccgagatgctgcgaacagagagctgccgccccaggtcgcccgccggacaggtggccgcggcgtccccgctcctgctgctgctgctgctgctcgcctggtgcgcgggcgcctgccgaggtgctccaatattacctcaaggattacagcctgaacaacagctacagttgtggaatgagatagatgatacttgttcgtcttttctgtccattgattctcagcctcaggcatccaacgcactggaggagctttgctttatgattatgggaatgctaccaaagcctcaggaacaagatgaaaaagataatactaaaaggttcttatttcattattcgaagacacagaagttgggcaagtcaaatgttgtgtcgtcagttgtgcatccgttgctgcagctcgttcctcacctgcatgagagaagaatgaagagattcagagtggacgaagaattccaaagtccctttgcaagtcaaagtcgaggatattttttattcaggccacggaatggaagaaggtcagcagggttcatttaaaatggatgccagctaattttccacagagcaatgctatggaatacaaaatgtactgacattttgttttcttctgaaaaaaatccttgctaaatgtactctgttgaaaatccctgtgttgtcaatgttctcagttgtaacaatgttgtaaatgttcaatttgttgaaaattaaaaaatctaaaaataaa

SEQ ID NO: 2 - protein, 174 aa, Homo sapiens
MLRTESCRPRSPAGQVAAASPLLLLLLLLAWCAGACRGAPILPQGLQPEQQLQLWNEIDDTCSSFLSIDSQPQASNALEELCFMIMGMLPKPQEQDEKDNTKRFLFHYSKTQKLGKSNVVSSVVHPLLQLVPHLHERRMKRFRVDEEFQSPFASQSRGYFLFRPRNGRRSAGFI

SEQ ID NO: 3 - DNA, 870 bp, Homo sapiens
  CDS (1)..(867)
atgtggaacgcgacgcccagcgaagagccggggttcaacctcacactggccgacctggactgggatgcttcccccggcaacgactcgctgggcgacgagctgctgcagctcttccccgcgccgctgctggcgggcgtcacagccacctgcgtggcactcttcgtggtgggtatcgctggcaacctgctcaccatgctggtggtgtcgcgcttccgcgagctgcgcaccaccaccaacctctacctgtccagcatggccttctccgatctgctcatcttcctctgcatgcccctggacctcgttcgcctctggcagtaccggccctggaacttcggcgacctcctctgcaaactcttccaattcgtcagtgagagctgcacctacgccacggtgctcaccatcacagcgctgagcgtcgagcgctacttcgccatctgcttcccactccgggccaaggtggtggtcaccaaggggcgggtgaagctggtcatcttcgtcatctgggccgtggccttctgcagcgccgggcccatcttcgtgctagtcggggtggagcacgagaacggcaccgacccttgggacaccaacgagtgccgccccaccgagtttgcggtgcgctctggactgctcacggtcatggtgtgggtgtccagcatcttcttcttccttcctgtcttctgtctcacggtcctctacagtctcatcggcaggaagctgtggcggaggaggcgcggcgatgctgtcgtgggtgcctcgctcagggaccagaaccacaagcaaaccgtgaaaatgctgggtgggtctcagcgcgcgctcaggctttctctcgcgggtcctatcctctccctgtgccttctcccttctctctga

SEQ ID NO: 4 - protein, 289 aa, Homo sapiens
MWNATPSEEPGFNLTLADLDWDASPGNDSLGDELLQLFPAPLLAGVTATCVALFVVGIAGNLLTMLVVSRFRELRTTTNLYLSSMAFSDLLIFLCMPLDLVRLWQYRPWNFGDLLCKLFQFVSESCTYATVLTITALSVERYFAICFPLRAKVVVTKGRVKLVIFVIWAVAFCSAGPIFVLVGVEHENGTDPWDTNECRPTEFAVRSGLLTVMVWVSSIFFFLPVFCLTVLYSLIGRKLWRRRRGDAVVGASLRDQNHKQTVKMLGGSQRALRLSLAGPILSLCLLPSL

SEQ ID NO: 5 - DNA, 4131 bp, Homo sapiens
  CDS (373)..(1626)
tcaagctcgccccgcgcagcccgagccgggctgggcgctgtcctcgggggcctggggaaccgcgcggtttggagatcggaggcacctggaacccgtggcaagcgccgagccgggagacagcccgaggaaccacgggttctggagctaggagccggaagctgggagtccggaggagagcggagcccggagcccggagcccggggcggcgcgtctgggtctggcgcttcccgactggacggcgcgcccgctggtcttcgccacgcgccctcccctgggctcgcgttcatcggtccccgcctgagacgcgcccactcctgcccggacttccagccccggaggcgccggacagagccgcggactccagcgcccaccatgcgcctcaacagctccgcgccgggaaccccgggcacgccggccgccgaccccttccagcgggcgcaggccggactggaggaggcgctgctggccccgggcttcggcaacgcttcgggcaacgcgtcggagcgcgtcctggcggcacccagcagcgagctggacgtgaacaccgacatctactccaaagtgctggtgaccgccgtgtacctggcgctcttcgtggtgggcacggtgggcaacacggtgacggcgttcacgctggcgcggaagaagtcgctgcagagcctgcagagcacggtgcattaccacctgggcagcctggcgctgtccgacctgctcaccctgctgctggccatgcccgtggagctgtacaacttcatctgggtgcaccacccctgggccttcggcgacgccggctgccgcggctactacttcctgcgcgacgcctgcacctacgccacggccctcaacgtggccagcctgagtgtggagcgctacctggccatctgccaccccttcaaggccaagaccctcatgtcccgaagccgcaccaagaagttcatcagcgccatctggctcgcctcggccctgctgacggtgcctatgctgttcaccatgggcgagcagaaccgcagcgccgacggccagcacgccggcggcctggtgtgcacccccaccatccacactgccaccgtcaaggtcgtcatacaggtcaacaccttcatgtccttcatattccccatggtggtcatctcggtcctgaacaccatcatcgccaacaagctgaccgtcatggtacgccaggcggccgagcagggccaagtgtgcacggtcgggggcgagcacagcacattcagcatggccatcgagcctggcagggtccaggccctgcggcacggcgtgcgcgtcctacgtgcagtggtcatcgcctttgtggtctgctggctgccctaccacgtgcggcgcctcatgttctgctacatctcggatgagcagtggactccgttcctctatgacttctaccactacttctacatggtgaccaacgcactcttctacgtcagctccaccatcaaccccatcctgtacaacctcgtctctgccaacttccgccacatcttcctggccacactggcctgcctctgcccggtgtggcggcgcaggaggaagaggccagccttctcgaggaaggccgacagcgtgtccagcaaccacaccctctccagcaatgccacccgcgagacgctgtactaggctgtgcgccccggaacgtgtccaggaggagcctggccatgggtccttgcccccgacagacagagcagcccccacccgggagccttgatgggggtcaggcagaggccagcctgcactggagtctgaggcctgggacccccccctcccaccccctaacccatgtttctcattagtgtctcccgggcctgtccccaactcctccccacccctcccccatctcctctttgaaagccagaacaagagagcgctcctctcccagataggaaaagggcctctaacaaggagaaattagtgtgcggcaaaaggcagttttctttgttctcagactaatggatggttccagagaaggaaatgaaatgtgctgggtggggccgggcctccggcggcccggctgctgttcccatgtccacatctctgaggcctgcaccccctctgtctagctcggggagtccagccccagtcccgcaggctccgtggctttgggcctcacgtgcagaccctgccatgcagacccatgcccccctcccccaggcagctccaagaaagctccctgactcgccccttcaggcctggcaagctgggggcccatcgccgtggggagtccctcccaccaccctcgccgcaggcagctgcagcccccagaggggaccacaagcccaaaaaggacaaaaatgggctggcctggaatggcccagaccccagcctcccctcctccctcccatcctcacccaggccaaggcccaggggctctgccaggacaccacatgggagggggctcaggcctcagcctcaagatcttcagctgtggcctctcgggctcggcagaagggacgccggatcaggggcctggtctccagcacctgcccgagtggccgtggccaggatggggtgcgcattccgtgtgctttgcttgtagctgtgcaggctgaggtctggagccaggcccagagctggcttcagggtggggccttgagaaggggaatgtgggacaggggcgatggtgcctggtctctgagtaagatgccaggtcccaggaactcaggcttcaggtgagaaggagcggtgtgtccaggcaccgctggccggcagccctgggctgaggcacagactcatttgtcaccttctggcggcggcagccctggccccggcctccaagcagttgaaaaagctggcgcctccttggtctctaggatccaggctccacagagcacatgactagccaggcccctggcttaagaaggtcgcctaagcctaagagaagacagtcccaggagaagctggccgggaccagccaggagctgggagccacaggaagcaaaagtcagccttttcttcaagggatttccctgtctcagagcagcctttgccccagggaaatgggctctgggctggctgcctgcaccggccatgtcgacccaggacccggacacctggtcttgggctgtgttcagccactttgccttctctggactcagtttccccgtctgagaaatgagagtcgaatgctacagtatctgcagtcgcttggatctggctgttgagttgacgggttccttgaaccccacaaaatccctctccaaccacaggacccttcggctcaccaagaacggggcccaggggagtcaggcctattcgctgcacttcctgccaaactttgcccccacaagcctggtcatcagccaggcagccctcccagtgcccaagggccaccaaccccagggaaacagggccagcacagaggggccttcctcccccacagagctcccatgacatagtctgctctgggcggaagagctttgctgccagccagggatgtccagaggtcggtgcagcccctatccctgctcaggagtgggctcagagtctagcaaatgctaaggcccctcaggctgggctctgaacgaggacctggactcagagccagacagggcagcctcagacccttctctggggctcctggaccttgggccataatttctgagcctcggtttccccatctaaggaacagatgtggtcgttccgccctctcagctggatgagactgtcctggaggatccaccccggaacagacagaacggtgtctctcaggatggtgctctgagagagggcagagtggatgccccactgccctagaccctcggtagacgtggggtctctggggcggggtctgtggctgtgactgaagtcggctttcccgttgatgtcttgatgctcctatctgtgcacttaccgtaggtagggacacgtgtccatgcaccacagacacacccacgacacctgatctcgtatcactagcttgcggccaggtcatgatgtggccccggaagctggccctgcgtgccatgagtgcgtcggtcatggagtccggagcccctgagccggcccctggtgacggcacagccctcacagctcaaacgcccacccccactcccaccatctgcaggtggtgaaaacaaaccccgtgtatctctcaataaaggtggccgaagggcctcgatgtgg

SEQ ID NO: 6 - protein, 418 aa, Homo sapiens
MRLNSSAPGTPGTPAADPFQRAQAGLEEALLAPGFGNASGNASERVLAAPSSELDVNTDIYSKVLVTAVYLALFVVGTVGNTVTAFTLARKKSLQSLQSTVHYHLGSLALSDLLTLLLAMPVELYNFIWVHHPWAFGDAGCRGYYFLRDACTYATALNVASLSVERYLAICHPFKAKTLMSRSRTKKFISAIWLASALLTVPMLFTMGEQNRSADGQHAGGLVCTPTIHTATVKVVIQVNTFMSFIFPMVVISVLNTIIANKLTVMVRQAAEQGQVCTVGGEHSTFSMAIEPGRVQALRHGVRVLRAVVIAFVVCWLPYHVRRLMFCYISDEQWTPFLYDFYHYFYMVTNALFYVSSTINPILYNLVSANFRHIFLATLACLCPVWRRRRKRPAFSRKADSVSSNHTLSSNATRETLY

SEQ ID NO: 7 - DNA, 22 bp, Artificial
  An artificially synthesized primer sequence for RT-PCR.
tgaagagattcagagtggacga

SEQ ID NO: 8 - DNA, 23 bp, Artificial
  An artificially synthesized primer sequence for RT-PCR.
actgagaacattgacaacacagg

SEQ ID NO: 9 - DNA, 22 bp, Artificial
  An artificially synthesized primer sequence for RT-PCR.
aagagggacagggacaagtagt

SEQ ID NO: 10 - DNA, 21 bp, Artificial
  An artificially synthesized primer sequence for RT-PCR.
atgccactgttactgcttcag

SEQ ID NO: 11 - DNA, 23 bp, Artificial
  An artificially synthesized primer sequence for RT-PCR.
ggctcttacaactcatgtaccca

SEQ ID NO: 12 - DNA, 24 bp, Artificial
  An artificially synthesized primer sequence for RT-PCR.
tgatacagagacatgaagtgagca

SEQ ID NO: 13 - DNA, 19 bp, Artificial
  An artificially synthesized primer sequence for RT-PCR.
tggtgtttgccttcatcct

SEQ ID NO: 14 - DNA, 20 bp, Artificial
  An artificially synthesized primer sequence for RT-PCR.
gaatcccagaagtctgaaca

SEQ ID NO: 15 - DNA, 19 bp, Artificial
  An artificially synthesized primer sequence for RT-PCR.
cttgggacaccaacgagtg

SEQ ID NO: 16 - DNA, 19 bp, Artificial
  An artificially synthesized primer sequence for RT-PCR.
aggacccgcgagagaaagc

SEQ ID NO: 17 - DNA, 20 bp, Artificial
  An artificially synthesized primer sequence for RT-PCR.
ggtctgtggctgtgactgaa

SEQ ID NO: 18 - DNA, 20 bp, Artificial
  An artificially synthesized primer sequence for RT-PCR.
gtttgagctgtgagggctgt

SEQ ID NO: 19 - DNA, 21 bp, Artificial
  An artificially synthesized primer sequence for RT-PCR.
tgagccctgaacaccagagag

SEQ ID NO: 20 - DNA, 21 bp, Artificial
  An artificially synthesized primer sequence for RT-PCR.
aaagccagatgagcgcttcta

SEQ ID NO: 21 - DNA, 22 bp, Artificial
  An artificially synthesized primer sequence for RT-PCR.
tcttcagcatgatgtgttgtgt

SEQ ID NO: 22 - DNA, 24 bp, Artificial
  An artificially synthesized primer sequence for RT-PCR.
tgagagattcatgaggaagtcttg

SEQ ID NO: 23 - DNA, 21 bp, Artificial
  An artificially synthesized primer sequence for RT-PCR.
ccctgacaacatcaactggtc

SEQ ID NO: 24 - DNA, 23 bp, Artificial
  An artificially synthesized primer sequence for RT-PCR.
gtccaccttcgcttttattgagt

SEQ ID NO: 25 - DNA, 22 bp, Artificial
  An artificially synthesized primer sequence for RT-PCR.
aaaaaggggatgcctagaactc

SEQ ID NO: 26 - DNA, 21 bp, Artificial
  An artificially synthesized primer sequence for RT-PCR.
ctttcagcacgtcaaggacat

SEQ ID NO: 27 - DNA, 23 bp, Artificial
  An artificially synthesized primer sequence for RT-PCR.
acacctacgaaggtacacatgac

SEQ ID NO: 28 - DNA, 23 bp, Artificial
  An artificially synthesized primer sequence for RT-PCR.
gctatttcagggtaaatggagtc

SEQ ID NO: 29 - DNA, 23 bp, Artificial
  An artificially synthesized primer sequence for RT-PCR.
cagagatggaggatgtcaataac

SEQ ID NO: 30 - DNA, 23 bp, Artificial
  An artificially synthesized primer sequence for rt-pcr.
catagcagctttaaagagacacg

SEQ ID NO: 31 - DNA, 21 bp, Artificial
  An artificially synthesized primer sequence for RT-PCR.
ccaccataacagtggagtggg

SEQ ID NO: 32 - DNA, 24 bp, Artificial
  An artificially synthesized primer sequence for RT-PCR.
cagttacaggtgtatgactgggag

SEQ ID NO: 33 - DNA, 23 bp, Artificial
  An artificially synthesized primer sequence for RT-PCR.
ctgaatacaacttcctgtttgcc

SEQ ID NO: 34 - DNA, 23 bp, Artificial
  An artificially synthesized primer sequence for RT-PCR.
gaccacagaattaccaaaactgc

SEQ ID NO: 35 - DNA, 22 bp, Artificial
  An artificially synthesized primer sequence for RT-PCR.
aaatagagcgtgaagatgccct

SEQ ID NO: 36 - DNA, 23 bp, Artificial
  An artificially synthesized primer sequence for RT-PCR.
ggcagctggcatcattaatactt

SEQ ID NO: 37 - DNA, 24 bp, Artificial
  An artificially synthesized primer sequence for RT-PCR.
gggttcttgttttatatacctggc

SEQ ID NO: 38 - DNA, 22 bp, Artificial
  An artificially synthesized primer sequence for RT-PCR.
gaattatggcagcaatcacaag

SEQ ID NO: 39 - DNA, 23 bp, Artificial
  An artificially synthesized primer sequence for RT-PCR.
ctggatgttgtgtgtatcgagag

SEQ ID NO: 40 - DNA, 22 bp, Artificial
  An artificially synthesized primer sequence for RT-PCR.
gtcttctgctggaaacatgccg

SEQ ID NO: 41 - DNA, 21 bp, Artificial
  An artificially synthesized primer sequence for RT-PCR.
gaggtgatagcattgctttcg

SEQ ID NO: 42 - DNA, 21 bp, Artificial
  An artificially synthesized primer sequence for RT-PCR.
caagtcagtgtacaggtaagc

SEQ ID NO: 43 - DNA, 32 bp, Artificial
  An artificially synthesized primer sequence for RT-PCR.
cgcggatccgcgatgctgcgaacagagagctg

SEQ ID NO: 44 - DNA, 32 bp, Artificial
  An artificially synthesized primer sequence for RT-PCR.
ccgctcgagcggaatgaaccctgctgaccttc

SEQ ID NO: 45 - protein, 19 aa, Homo sapiens
GGSQRALRLSLAGPILSLC

SEQ ID NO: 46 - DNA, 19 bp, Artificial
  An artificially synthesized target sequence for siRNA
gaagcagcacgacttcttc

SEQ ID NO: 47 - DNA, 19 bp, Artificial
  An artificially synthesized target sequence for siRNA
cgtacgcggaatacttcga

SEQ ID NO: 48 - DNA, 19 bp, Artificial
  An artificially synthesized target sequence for siRNA
gcgcgctttgtaggattcg

SEQ ID NO: 49 - DNA, 19 bp, Artificial
  An artificially synthesized target sequence for siRNA
gagattcagagtggacgaa

SEQ ID NO: 50 - DNA, 19 bp, Artificial
  An artificially synthesized target sequence for siRNA
cctctacctgtccagcatg

SEQ ID NO: 51 - DNA, 19 bp, Artificial
  An artificially synthesized target sequence for siRNA
gctggtcatcttcgtcatc

SEQ ID NO: 52 - DNA, 19 bp, Artificial
  An artificially synthesized target sequence for siRNA
gttcatcagcgccatctgg

SEQ ID NO: 53 - DNA, 19 bp, Artificial
  An artificially synthesized target sequence for siRNA
ggtcgtcatacaggtcaac

SEQ ID NO: 54 - DNA, 19 bp, Artificial
  An artificially synthesized target sequence for siRNA
gcagcagaaacgaccgaat

SEQ ID NO: 55 - protein, 16 aa, Artificial
  An artificially synthesized c-myc-His-epitope sequence
LDEESILKQEHHHHHH

SEQ ID NO: 56 - DNA, 32 bp, Artificial
  An artificially synthesized primer sequence for RT-PCR.
ggaattccatgtggaacgcgacgcccagcgaa

SEQ ID NO: 57 - DNA, 36 bp, Artificial
  An artificially synthesized primer sequence for RT-PCR.
cgcggatccgcggagagaagggagaaggcacaggga

SEQ ID NO: 58 - DNA, 36 bp, Artificial
  An artificially synthesized primer sequence for RT-PCR.
ggaattccatgcgcctcaacagctccgcgccgggaa

SEQ ID NO: 59 - DNA, 39 bp, Artificial
  An artificially synthesized primer sequence for RT-PCR.
cgcggatccgcggtacagcgtctcgcgggtggcattgct

SEQ ID NO: 60 - protein, 16 aa, Artificial
  Artificially synthesized domain-peptide of GHSR
GVEHENGTDPWDTNEC

(19) United States
(12) Patent Application Publication
(10) Pub. No.: US 2009/0311685 A1
(43) Pub. Date: Dec. 17, 2009

(54) NMU-GHSR1b/NTSR1 oncogenic signaling pathway as a therapeutic target for lung cancer

(75) Inventors: Yusuke Nakamura, Tokyo (JP); Yataro Daigo, Tokyo (JP); Shuichi Nakatsuru, Kanagawa (JP)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Oncotherapy Science, Inc, Kawasaki-shi (JP)

(21) Appl. No.: 12/297,764

(22) PCT Filed: Apr. 18, 2007
(86) PCT No.: PCT/JP2007/058893

§ 371 Date: Feb. 13, 2009

Related U.S. Application Data
(60) Provisional application No. 60/793,977, filed on Apr. 20, 2006.

(51) Int. Cl. C12Q 1/68; G01N 33/53

(52) U.S. Cl. 435/6; 435/7.2

(57) ABSTRACT

The present invention relates to a method of and a kit for assessing the prognosis of lung cancer by detecting the expression level of the neuromedin U (NMU) gene in a patient-derived biological sample. The method and kit are particularly preferred for assessing the prognosis of non-small cell lung cancer (NSCLC). Furthermore, the present invention relates to a method of screening for a therapeutic agent for cancer, in particular, lung cancer, by detecting compounds that inhibit the binding of the NMU protein with the heterodimer of growth hormone secretagogue receptor 1b (GHSR1b) and neurotensin receptor 1 (NTSR1).

This application claims the benefit of U.S. Provisional Application Ser. No. 60/793,977 filed Apr. 20, 2006, the contents of which are hereby incorporated by reference in their entirety.

### FIELD OF THE INVENTION

The present invention relates to the field of biological science, more specifically to the field of cancer research. More particularly, the present invention relates to a method of assessing or determining the prognosis of lung cancer which was accomplished by the discovery that neuromedin U (NMU) gene serves as a prognostic marker of lung cancer. Furthermore, the present invention relates to a kit that can be used for assessing or determining the prognosis of lung cancer. Moreover, the present invention relates to a method of identifying and/or screening for a therapeutic agent for cancer, in particular, lung cancer, based on the discovery that growth hormone secretagogue receptor 1b (GHSR1b) and neurotensin receptor 1 (NTSR1), which were known to bind to the NMU protein, form a heterodimer complex.

### BACKGROUND OF THE INVENTION

Lung cancer is one of the most common causes of cancer-death worldwide, and non-small cell lung cancer (NSCLC) accounts for nearly 80% of those cases (Greenlee R. T. et al. (2001) CA Cancer J. Clin. 51: 15-36). Many genetic alterations associated with development and progression of lung cancer have been reported, but the precise molecular mechanisms remain unclear. Over the last decade newly developed cytotoxic agents including paclitaxel, docetaxel, gemcitabine, and vinorelbine have emerged to offer multiple therapeutic choices for patients with advanced NSCLC. However, each of the new regimens can provide only modest survival benefits compared with conventional cisplatin-based therapies (Schiller J. H. et al. (2002) N. Engl. J. Med. 10;346: 92-8). Hence, new therapeutic strategies such as molecular-targeted agents, antibodies, and cancer vaccines are eagerly awaited.

Systematic analysis of expression levels of thousands of genes on cDNA microarrays is an effective approach for identifying unknown molecules involved in pathways of carcinogenesis. Such genes and their products can be investigated as potential targets for development of novel therapeutics and diagnostics (Kikuchi T. et al. (2003) Oncogene 10;22: 2192-205; Kato T. et al. (2005) Cancer Res. 65: 5638-46; Ishikawa N. et al. (2005) Cancer Res. 65: 9176-84). The inventors have identified potential molecular targets for diagnosis and treatment of lung cancer by analyzing genome-wide expression profiles of NSCLC cells on a cDNA microarray containing 23,040 genes, after enrichment of tumor cells from 37 cancer tissues by laser-capture microdissection (Kikuchi T. et al. (2003) Oncogene 10;22: 2192-205). To verify the biological and clinicopathological significance of the respective gene products, tumor-tissue microarray analysis of clinical lung-cancer materials have been performed by the present inventors (Ishikawa et al. (2004) Clin. Cancer Res. 10: 8363-70; Ishikawa et al. (2005) Cancer Res. 65: 5638-46; Furukawa et al. (2005) Cancer Res. 65: 7102-10). During the course of those studies, the gene encoding neuromedin U (NMU) (GenBank Accession No. NM_{—}006681; SEQ ID NOs: 1 and 2) was frequently observed to be over-expressed in primary NSCLCs.

NMU is a neuropeptide that was first isolated from porcine spinal cord. It has potent activity on smooth muscle (Minamino et al. (1985) Biophys. Res. Commun. 130: 1078-85; Minamino et al. (1988) Biochem. Biophys. Res. Commun. 156: 355-60; Domin et al. (1986) Biochem. Biophys. Res. Commun. 140: 1127-34; Domin et al. (1988) J. Biol. Chem. 264: 20881-5; Conlon et al. (1988) J. Neurochem. 51: 988-91; O'Harte et al. (1991) Peptides 12: 809-12; Kage et al. (1991) Regul. Pept. 33: 191-8; Austin et al. (1994) J Mol Endocrinol. 12: 257-63; Fujii et al. (2000) J. Biol. Chem. 275: 21068-74), and in mammalian species NMU is distributed predominantly in the gastrointestinal tract and central nervous system (Minamino et al. (1985) Biochem. Biophys. Res. Commun. 130: 1078-85; Howard et al. (2000) Nature 406: 70-4; Funes et al. (2002) Peptides 23: 1607-15). Peripheral activities of NMU include stimulation of smooth muscle, alteration of ion transport in the gut, and regulation of feeding (Howard et al. (2000) Nature 406: 70-4).

A C-terminal asparaginamide structure and the C-terminal hepatapeptide core of NMU protein are essential for its contractile activity in smooth-muscle cells (Austin et al. (1995) J. Mol. Endocrinol. 14: 157-69; Westfall et al. (2002) J. Pharmacol. Exp. Ther. 301: 987-92). Recent studies have indicated that NMU acts at the hypothalamic level to inhibit food intake, and therefore, this protein might be a physiological regulator of feeding and body weight (Maggi et al. (1990) Br. J. Pharmacol. 99: 186-8; Howard et al. (2000) Nature 406: 70-4; Ivanov et al. (2002) Endocrinology 143: 3813-21; Wren et al. (2002) Endocrinology 143: 4227-34; Hanada et al. (2004) Nat. Med. 10: 1067-73). NMU is also reported to be expressed in several types of human tumors (Steel et al. (1988) Endocrinology 122: 270-82; Shetzline et al. (2004) Blood 104: 1833-40; Euer et al. (2005) Oncol. Rep. 13: 375-87).

Neuropeptides function peripherally as paracrine and autocrine factors to regulate diverse physiologic processes and act as neurotransmitters or neuromodulators in the nervous system. In general, the receptors which mediate signaling by binding neuropeptides are members of the G protein-coupled receptor (GPCR) superfamily which peptides have seven transmembrane-spanning domains. Two known receptors for NMU, NMU1R (FM3/GPR66) and NMU2R (FM4), show a high degree of homology to other neuropeptide receptors such as growth hormone secretagogue receptor (GHSR) and neurotensin receptor 1 (NTSR1), for which the corresponding known ligands are ghrelin (GHRL) and nerotensin (NTS), respectively. Each of these two receptors has seven predicted alpha-helical transmembrane domains containing highly conserved motifs, as do other members of the rhodopsin GPCR family (Fujii et al. (2000) J. Biol. Chem. 275: 21068-74; Howard et al. (2000) Nature 406: 70-4; Funes et al. (2002) Peptides 23: 1607-15).

Recent acceleration in the identification and characterization of novel molecular targets for cancer therapy has stimulated considerable interest on the development of new types of anti-cancer agents (Kelly et al. (2001) J. Clin. Oncol. 19: 3210-8; Schiller et al. (2002) N. Engl. J. Med. 346: 92-8). Although advances have been made in the development of molecular-targeting drugs for cancer therapy, the range of responsive tumor types and the effectiveness of such treatments are still very limited (Ranson M. et al. (2002) J. Clin. Oncol. 20: 2240-50; Blackledge G & Averbuch S. (2004) Br. J. Cancer 90: 566-72). Hence, the development of novel anti-cancer agents that are highly specific to malignant cells and evoke minimal or no adverse reactions is urgently required in the art. A powerful strategy toward such goal combines screening of up-regulated genes in cancer cells on the basis of expression-profile information with a high-throughput functional analysis. The approach of functional analysis by the present inventors includes examination of loss-of-function phenotypes using RNAi technology, investigating the effect of gene product on growth and cell-mobility, identifying proteins that interact with the gene product, and analyzing tissue microarrays prepared from hundreds of clinical samples (ononen J. et al. (1998) Nat. Med. 4:844-7; Sauter G et al. (2003) Nat. Rev. Drug Discov. 2: 962-72).

By pursuing such technology, the inventors have been able to show that the NMU gene is overexpressed in a great majority of clinical NSCLC samples and cell lines (WO 2004/031413). Further, it was revealed that the growth of NSCLC cells that overexpress endogenous NMU can be inhibited by anti-NMU antibody and siRNA against NMU; that NMU binds to the neuropeptide GPCRs, growth hormone secretagogue receptor 1b (GHSR1b), and NTSR1; that the NMU ligand-receptor system activates Homo sapiens forkhead box M1 (FOXM1); and that, in addition to NMU, GHSR1, NTSR1, and FOXM1 are overexpressed in NSCLC cells (WO 2004/031413).

GHSR is a known receptor of GHRL, a recently identified 28-amino-acid peptide capable of stimulating the release of pituitary growth hormone and appetite in human (Kojima et al. (1999) Nature 402: 656-60; Kim et al. (2001) Clin. Endocrinol. 54: 759-768; Lambert et al. (2001) Proc. Natl. Acad. Sci. USA 98: 4652-7; Petersenn et al. (2001) Endocrinology 142: 2649-59). Of the two transcripts known to be receptors for GRL, GHSR1a and GHSR1b, overexpression of only GHSR1b was detected in NSCLC tissues and cell lines. In NSCLC, GHRL was not significantly expressed in examined cell lines. Therefore, the present inventors suspected that GHSR1b could have a growth-promoting function in lung tumors through the binding to NMU, but not to GHRL. Interestingly, it was reported that GHRL and GHSR1b, but not GHSR1a genes were overexpressed in erythroleukemic HEL cells, whose proliferation was regulated by des-acyl GHRL in an autocrine manner (Vriese et al. (2005) Endocrinology 146: 1514-22).

NTSR1 is one of three receptors of NTS, a brain and gastrointestinal peptide that fulfils many central and peripheral functions (Heasley et al. (2001) Oncogene 20: 1563-9). NTS modulates transmission of dopamine and secretion of pituitary hormones, and exerts hypothermic and analgesic effects in the brain while it functions as a peripheral hormone in the digestive tract and cardiovascular system. Others have reported that NTS is produced and secreted in several human cancers, including SCLCs (Heasley et al. (2001) Oncogene 20: 1563-9). The present inventors detected the expression of NTS in four of 15 examined NSCLC cell lines, but the expression pattern of NTS was not necessarily concordant with that of NMU or NTSR1. Therefore, it was assumed that NTS might contribute to the growth of NSCLC through NTSR1 or other receptor(s) in a small subset of NSCLCs.

Heterodimerization of receptors has been shown to contribute to both ligand-binding affinity and signaling efficacy of GPCRs (Bouvier (2001) Nat. Rev. Neurosci. 2: 274-86; Devi (2001) Trends Pharmacol. Sci. 22: 532-7). Heterodimers can be formed by receptors for various ligands/transmitters; for example, GPCRs for angiotensin and bradykinin (AbdAlla et al. (2000) Nature 407: 94-8), those for dopamine and adenosine (Gines et al. (2000) Proc. Natl. Acad. Sci. USA 97: 8606-11), or those for opioid and adrenergic ligands (Rocheville et al. (2000) Science 288: 154-7). Moreover, it has been reported that co-expression of GHSR1a and GHSR1b resulted in an attenuation of the signaling capability of GHSR1a, suggesting that GHSR1b possibly interacts with GHSR1a through receptor heterodimerization (Chan et al. (2004) Mol. Cell Endocrinol. 214: 81-95).

FOXM1, a member of the forkhead gene family, was known to be overexpressed in several types of human cancers (Teh et al. (2002) Cancer Res. 62: 4773-80; van den Boom et al. (2003) Am. J. Pathol. 163: 1033-43; Kalinichenko et al. (2004) Genes Dev. 18: 830-50). The “forkhead” gene family, originally identified in Drosophila, comprises transcription factors with a conserved 100-amino acids DNA-binding motif, and has been shown to play important roles in regulating the expression of genes involved in cell growth, proliferation, differentiation, longevity, and transformation. Reported assays using a human osteosarcoma cell line U20S demonstrated that exogenous FOXM1-mediated stimulation of hepatocyte DNA replication was associated with increased expression of CCND1 and CCNA1 (Wang et al. (2001) Proc. Natl. Acad. Sci. USA 98: 11468-73). The report suggested that these cyclin genes are possible transcription targets of FOXM1 transcription factor and that FOXM1 controls the transcription network of genes that are essential for cell division and exit from mitosis.

### SUMMARY OF THE INVENTION

According to the present invention, a method for assessing or determining the prognosis of a patient with lung cancer is provided. Specifically, the expression level of the neuromedin U (NMU) gene is determined in a biological sample, such as sputum or blood, derived from the patient and compared to a control (expression) level of the gene. Herein, an increase of the expression level of the gene compared to a good prognosis control level indicates poor prognosis, i.e., poor survival of the patient. Such an increase may, for example, at least 10% greater than the control level. The present method is particularly suited for assessing or determining the prognosis of non-small cell lung cancer (NSCLC).

In an embodiment of the present invention, the expression level of the NMU gene in the biological sample may be determined by detecting the amount of NMU mRNA or the amount or activity of the NMU protein. For example, the amount of NMU mRNA may be determined by hybridization of a probe to the mRNA, e.g., on a DNA array. Alternatively, the amount of the NMU protein may be detected via the use of an anti-NMU protein antibody.

Further, the expression level of other lung-cancer associated genes may also be determined in the present invention, to improve the accuracy of the assessment.

Furthermore, according to an aspect of the present invention, a kit for assessing or determining the prognosis of a patient with lung cancer is provided. Specifically, the kit comprises a reagent for detecting the amount of NMU mRNA or the amount or activity of the NMU protein which correlates to the expression level of the NMU gene. According to a favorable aspect of the present invention, the kit comprises an antibody against the NMU protein.

In addition, the present invention provides a method of identifying or screening for a compound that inhibits the signal transduction by the heterodimer consisting of the growth hormone secretagogue receptor 1b (GHSR1b) and the neurotensin receptor 1 (NTSR1). Specifically, the method is performed by (1) contacting the heterodimer of GHSR1b and NTSR1, or a functional equivalent thereof with the NMU protein in the existence of a test compound; (2) detecting the signal transduction by the heterodimer and the NMU protein; and (3) selecting the test compound that inhibits the signal transduction by the heterodimer and the NMU protein. A compound that is identified or screened through such a method is expected to be useful for treating or preventing lung cancer, in particular NSCLC.

According to an aspect of the invention, a heterodimer that is expressed on the surface of a living cell is used in the method.

When the heterodimer is expressed on the surface of a living cell, the signal transduction by the heterodimer and the NMU protein is detected, for example, by:
(a) detecting the concentration of CAMP in the cell;
(b) detecting the activation of adenylate cyclase;
(c) detecting the activation of protein kinase A (PKA);
(d) detecting the expression of NMU target genes including FOXM1, GCDH, CDK5RAP1, LOC134145, and NUP188;
(e) detecting the change in subcellular localization of the heterodimer including ligand-induced internalization;
(f) detecting cell proliferation, transformation, or any other oncogenic phenotype of the cell; and
(g) detecting apoptosis of the cell.

These and other objects and features of the invention will become more fully apparent when the following detailed description is read in conjunction with the accompanying figures and examples. However, it is to be understood that both the foregoing summary of the invention and following detailed description are of preferred embodiments, and not restrictive of the invention or other alternate embodiments of the invention.

### BRIEF DESCRIPTION OF THE DRAWINGS

FIG. 1 depicts a graph showing NMU expression in primary lung cancers and cell lines. Kaplan-Meier analysis were performed for determining tumor-specific survival according to NMU expression in patients with NSCLC (p=0.036 by the Log-rank test).

FIG. 2 depicts the result of immunoprecipitation which shows the characteristic of the GHSR1b/NTSR1 heterodimer and its co-internalization as a cognate receptor of NMU. Cell lysates from COS-7 cells that transiently express the FLAG-tagged GHSR1b, and those co-expressing both the FLAG-tagged GHSR1b and NTSR1 were immunoprecipitated with anti-FLAG antibody, subjected for SDS-PAGE, and then immunoblotted with anti-FLAG antibody (A), with anti-NTSR1 antibody (B), or with anti-GHSR antibody (C). The arrows indicate the monomer, heterodimer, and homodimers of the receptors. The molecular weight (kDa) markers are indicated on the left side of individual panels. Non-specific immunoreactive protein band detected by anti-FLAG antibody is indicated with asterisks.

FIG. 3 depicts the result of experiments showing the relationship between the expression levels of GHSR1b/NTSR1 and intracellular cAMP production by NMU-25 in lung-cancer cell lines. The expression levels of receptors in LC319 (A), RERF-LC-AI (B), NCI-H358 (C), and SK-MES-1 (D) cells were detected by semiquantitative RT-PCR analysis. Dose-response curves of intracellular cAMP production by NMU-25 treatment (3 to 50 μM) in individual cell lines are shown. All experiments were done in triplicate.

### DETAILED DESCRIPTION OF THE INVENTION

According to a pervious study by the present inventors, using a genome-wide cDNA microarray, neuromedin U (NMU) was identified as a specifically up-regulated gene in non-small cell lung cancer (NSCLC) (WO 2004/031413). The treatment of NSCLC cells with siRNA against NMU was shown not only to suppress the expression of NMU but also inhibit the growth of the cells (WO 2004/031413). Concordantly, the growth of NSCLC cells that overexpress endogenous NMU was also significantly inhibited by anti-NMU antibody (WO 2004/031413). Furthermore, two G protein-coupled receptors, growth hormone secretagogue receptor 1b (GHSR1b) and neurotensin receptor 1 (NTSR1) were also found to be overexpressed in NSCLC cells and each were identified to interact with NMU, individually (WO 2005/090603).

Through a further study in relation with NMU, a significant increase in the sub-G1 fraction of NSCLC cells transfected with siRNA against NMU suggested that blocking the autocrine NMU-signaling pathway could induce apoptosis. The inventors also found other evidence supporting the significance of this pathway in carcinogenesis, e.g., addition of NMU into the medium promoted the growth of COS-7 cells in a dose-dependent manner; and the addition of anti-NMU antibody into the culture medium inhibited this NMU-enhanced cell growth, possibly by neutralizing NMU activity. Moreover, the growth of NSCLC cells that endogenously overexpress NMU was significantly inhibited by anti-NMU antibody. The expression of NMU resulted in significant promotion of cell invasion in an in vitro assay.

In addition, the present inventors newly discovered that NMU expression is significantly associated with poorer prognosis of NSCLC patients. Clinicopathological evidence obtained through tissue-microarray experiments demonstrated that NSCLC patients with tumors expressing NMU showed shorter cancer-specific survival periods than those with negative NMU expression. The result obtained by in vitro and in vivo assays strongly suggested that overexpressed NMU is likely to be an important growth factor and might be associated with cancer cell invasion, functioning in an autocrine manner, and that screening molecules targeting the NMU-receptor growth-promoting pathway should be a promising therapeutic approach for treating or preventing lung cancers. Since NMU is a secreted protein and most of the clinical NSCLC samples used for the analyses by the present inventors were from patients of early and operable stage of carcinogenesis, NMU might also serve as a biomarker for diagnosis of early-stage lung cancer as well as an indicator for a highly malignant phenotype of lung-cancer cells, in combination with fiberscopic transbronchial biopsy (TBB) or blood tests.

Furthermore, through the present study, it was revealed that the receptors GHSR1b and NTSR1 not only interact with NMU individually but also interact with each other forming a heterodimer complex that functions as an NMU receptor. According to the experiments by the present inventors, the majority of the cancer cell lines and clinical NSCLCs that expressed NMU also expressed GHSR1b and NTSR1, indicating that these ligand-receptor interactions are involved in a pathway that is central to the growth-promoting activity of NMU in NSCLCs. GHSR1b and NTSR1 were also expressed in COS-7 cells used to examine the growth and invasion effect of NMU, and the obtained data demonstrated the importance of these two receptors for oncogenesis.

Moreover, this receptor was shown to induce, upon the binding of NMU (or NMU-25), the generation of second messenger, cAMP, to activate its downstream genes including transcription factors and cell cycle regulators. Elevated cAMP levels were generally observed via the activation of adenylate cyclase, which activated protein kinase A (PKA). It was reported that GHRL did not displace ¹²⁵I-labeled rat NMU-binding to NMU1R-expressing cells when tested at concentrations up to 10 MM (Kojima et al. (1999) Nature 402: 656-60). However, GHRL or NTS competitively inhibited NMU-induced cAMP production in NSCLC cells. Moreover, in the present application, biochemical and physiological evidence supporting the internalization and heterodimerization of the two neuropeptide GPCRs, GHSR1b and NTSR1, are provided (Example; RESULTS(5)). These results independently show that NMU stimulates NSCLC cell proliferation by a pathway through GHSR1b-NTSR1 heterodimer whose function is quite different from the two known NMU-receptors, NMU1R and NMU2R. Taking the hitherto reports and the newly obtained results by the present inventors, NMU is shown to affect the growth of NSCLC cells through the activation of the cAMP/PKA signaling pathway through the binding to the GHSR1b/NTSR1 heterodimer, which is coupled with a G protein of the Gs subfamily.

In addition, the treatment of NSCLC cells with siRNAs for GHSR, NTSR1, or one of their downstream genes, forkhead box M1 (FOXM1), was demonstrated to suppress the expression of those genes and the growth of NSCLC cells, and induces apoptosis in cancer cells. To predict transcriptional regulation of the FOXM1 gene by cAMP-response element (CRE)-binding protein, the CRE-like sequence was screened within a 1-kb upstream region of the putative transcription start sequence (TSS) using computer prediction program and found that the region contains three CRE-like elements. Moreover, it should be noted that luciferase reporter gene assay suggested that two of the CRE-like sequences are essential for effective augmentation of FOXM1 promoter activity following NMU stimulation (unpublished data). It is speculated that the CRE-binding proteins phosphorylated by PKA might be directly responsible for the regulation of FOXM1 expression.

In summary, the present inventors have discovered that GHSR1b and NTSR1, which respectively were known to bind to NMU individually, form a GPCR heterodimer which as a whole serves as a functional receptor of NMU. Furthermore, it was discovered that NMU and this newly revealed heterodimer are not only overexpressed in the great majority of lung cancers, but also are essential for an autocrine growth-promoting pathway that activates various downstream genes including FOXM1, which is a transcription factor. Thus, targeting the NMU ligand-receptor signaling pathway is a useful new therapeutic strategy for the treatment of lung-cancer patients, i.e., NMU and its downstream molecules can be used as targets for the development of novel therapeutic drugs and diagnostic markers.

### I. Definitions

The words “a”, “an”, and “the” used herein mean “at least one” unless otherwise specifically indicated.

The terms “polypeptide”, “peptide”, and “protein” are used interchangeably herein to refer to a polymer of amino acid residues. The terms apply to amino acid polymers in which one or more amino acid residue is a modified residue, or a non-naturally occurring residue, such as an artificial chemical mimetic of a corresponding naturally occurring amino acid, as well as to naturally occurring amino acid polymers.

The term “amino acid” refers to naturally occurring and synthetic amino acids, as well as amino acid analogs and amino acid mimetics that similarly functions to the naturally occurring amino acids. Naturally occurring amino acids are those encoded by the genetic code, as well as those modified after translation in cells (e.g., hydroxyproline, Y-carboxyglutamate, and O-phosphoserine). The phrase “amino acid analog” refers to compounds that have the same basic chemical structure (an a carbon bound to a hydrogen, a carboxy group, an amino group, and an R group) as a naturally occurring amino acid but have a modified R group or modified backbones (e.g., homoserine, norleucine, methionine, sulfoxide, methionine methyl sulfonium). The phrase “amino acid mimetic” refers to chemical compounds that have different structures but similar functions to general amino acids.

Amino acids may be referred to herein by their commonly known three letter symbols or the one-letter symbols recommended by the IUPAC-IUB Biochemical Nomenclature Commission.

The terms “gene”, “polynucleotides”, “nucleotides” and “nucleic acids” are used interchangeably unless otherwise specifically indicated and are similarly to the amino acids referred to by their commonly accepted single-letter codes.

Unless otherwise defined, all technical and scientific terms used herein have the same meaning as commonly understood by one of ordinary skill in the art to which this invention belongs. In case of conflict, the present specification, including definitions, will control.

### II. Method for Assessing the Prognosis of Lung Cancer

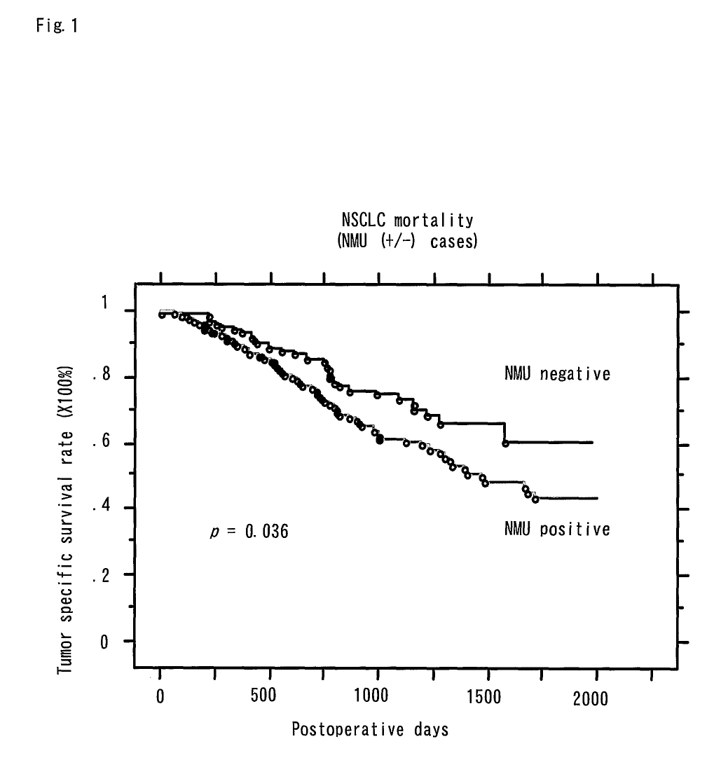

According to the present invention, it was newly discovered that NMU expression is significantly associated with poorer prognosis of NSCLC patients (see FIG. 1). Thus, the present invention provides a method for assessing or determining the prognosis of a patient with lung cancer, in particular, NSCLC, by detecting the expression level of the NMU gene in a biological sample of the patient; comparing the detected expression level to a control level; and determining a increased expression level to the control level as indicative of poor prognosis (poor survival).

Herein, the term “prognosis” refers to a forecast as to the probable outcome of the disease as well as the prospect of recovery from the disease as indicated by the nature and symptoms of the case. Accordingly, a less favorable, negative, poor prognosis is defined by a lower post-treatment survival term or survival rate. Conversely, a positive, favorable, or good prognosis is defined by an elevated post-treatment survival term or survival rate.

In the context of the present invention, the phrase “assessing (or determining) the prognosis” is intended to encompass predictions and likelihood analysis of lung cancer, progression, particularly NSCLC recurrence, metastatic spread and disease relapse. The present method for assessing or determining prognosis is intended to be used clinically in making decisions concerning treatment modalities, including therapeutic intervention, diagnostic criteria such as disease staging, and disease monitoring and surveillance for metastasis or recurrence of neoplastic disease.

The patient-derived biological sample used for the method may be any sample derived from the subject to be assessed so long as the NMU gene can be detected in the sample. Preferably, the biological sample comprises a lung cell (a cell obtained from the lung). Furthermore, the biological sample includes bodily fluids such as sputum, blood, serum, or plasma. Moreover, the sample may be cells purified from a tissue. The biological samples may be obtained from a patient at various time points, including before, during, and/or after a treatment.

According to the present invention, it was shown that the higher the expression level of the NMU gene measured in the patient-derived biological sample, the poorer the prognosis for post-treatment remission, recovery, and/or survival and the higher the likelihood of poor clinical outcome. Thus, according to the present method, the “control level” used for comparison may be, for example, the expression level of the NMU gene detected before any kind of treatment in an individual or a population of individuals who showed good or positive prognosis of NSCLC after the treatment, which herein will be referred to as “good prognosis control level”. Alternatively, the “control level” may be the expression level of the NMU gene detected before any kind of treatment in an individual or a population of individuals who showed poor or negative prognosis of NSCLC after the treatment, which herein will be referred to as “poor prognosis control level”. The “control level” is a single expression pattern derived from a single reference population or from a plurality of expression patterns. Thus, the control level may be determined based on the expression level of the NMU gene detected before any kind of treatment in a patient of NSCLC, or a population of the patients whose disease state (good or poor prognosis) is known. It is preferred, to use the standard value of the expression levels of the NMU gene in a patient group with a known disease state. The standard value may be obtained by any method known in the art. For example, a range of mean±2 S.D. or mean±3 S.D. may be used as standard value.

The control level may be determined at the same time with the test biological sample by using a sample(s) previously collected and stored before any kind of treatment from lung cancer patient(s) (control or control group) whose disease state (good prognosis or poor prognosis) are known.

Alternatively, the control level may be determined by a statistical method based on the results obtained by analyzing the expression level of the NMU gene in samples previously collected and stored from a control group. Furthermore, the control level can be a database of expression patterns from previously tested cells. Moreover, according to an aspect of the present invention, the expression level of the MU gene in a biological sample may be compared to multiple control levels, which control levels are determined from multiple reference samples. It is preferred to use a control level determined from a reference sample derived from a tissue type similar to that of the patient-derived biological sample.

According to the present invention, a similarity in the expression level of the NMU gene to the good prognosis control level indicates a more favorable prognosis of the patient and an increase in the expression level to the good prognosis control level indicates less favorable, poorer prognosis for post-treatment remission, recovery, survival, and/or clinical outcome. On the other hand, a decrease in the expression level of the NMU gene to the poor prognosis control level indicates a more favorable prognosis of the patient and a similarity in the expression level to the poor prognosis control level indicates less favorable, poorer prognosis for post-treatment remission, recovery, survival, and/or clinical outcome.

An expression level of the NMU gene in a biological sample can be considered altered when the expression level differs from the control level by more than 1.0, 1.5, 2.0, 5.0, 10.0, or more fold. Alternatively, an expression level of the NMU gene in a biological sample can be considered altered, when the expression level is increased or decreased relative to the control level at least 10%, 20%, 30%, 40%, 50%, 60%, 80%, 90%, or more.

The difference in the expression level between the test biological sample and the control level can be normalized to a control, e.g., housekeeping gene. For example, polynucleotides whose expression levels are known not to differ between the cancerous and non-cancerous cells, including those coding for β-actin, glyceraldehyde 3-phosphate dehydrogenase, and ribosomal protein P1, may be used to normalize the expression levels of the NMU gene.

The expression level may be determined by detecting the gene transcript in the patient-derived biological sample using techniques well known in the art. The gene transcripts detected by the present method include both the transcription and translation products, such as mRNA and protein.

For instance, the transcription product of the MU gene can be detected by hybridization, e.g., Northern blot hybridization analyses, that use an NMU gene probe to the gene transcript. The detection may be carried out on a chip or an array. The use of an array is preferable for detecting the expression level of a plurality of genes including the NMU gene. As another example, amplification-based detection methods, such as reverse-transcription based polymerase chain reaction (RT-PCR) which use primers specific to the NMU gene may be employed for the detection (see Example). The NMU gene-specific probe or primers may be designed and prepared using conventional techniques by referring to the whole sequence of the NMU gene (SEQ ID NO: 1). For example, the primers (SEQ ID NOs: 7 and 8; and SEQ ID NOs: 43 and 44) used in the Example may be employed for the detection by RT-PCR, but the present invention is not restricted thereto.

Specifically, a probe or primer used for the present method hybridizes under stringent, moderately stringent, or low stringent conditions to the mRNA of the NMU gene. As used herein, the phrase “stringent (hybridization) conditions” refers to conditions under which a probe or primer will hybridize to its target sequence, but to no other sequences. Stringent conditions are sequence-dependent and will be different under different circumstances. Specific hybridization of longer sequences is observed at higher temperatures than shorter sequences. Generally, the temperature of a stringent condition is selected to be about 5° C. lower than the thermal melting point (Tₘ) for a specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30° C. for short probes or primers (e.g., 10 to 50 nucleotides) and at least about 60° C. for longer probes or primers. Stringent conditions may also be achieved with the addition of destabilizing agents, such as formamide.

Alternatively, the translation product may be detected for the assessment of the present invention. For example, the quantity of the NMU protein may be determined. A method for determining the quantity of the protein as the translation product includes immunoassay methods that use an antibody specifically recognizing the NMU protein. The antibody may be monoclonal or polyclonal. Furthermore, any fragment or modification (e.g., chimeric antibody, scFv, Fab, F(ab′)₂, Fv, etc.) of the antibody may be used for the detection, so long as the fragment retains the binding ability to the NMU protein. Methods to prepare these kinds of antibodies for the detection of proteins are well known in the art, and any method may be employed in the present invention to prepare such antibodies and equivalents thereof.

As another method to detect the expression level of the NMU gene based on its translation product, the intensity of staining may be observed via immunohistochemical analysis using an antibody against NMU protein. Namely, the observation of strong staining indicates increased presence of the NMU protein and at the same time high expression level of the NMU gene. NSCLC tissue can be preferably used as a test material for immunohistochemical analysis.

Furthermore, the translation product may be detected based on its biological activity. Specifically, the NMU protein is known to bind to GHSR1b and NTSR1, and thus the expression level of the NMU gene can be detected by measuring the binding ability to GHSR1b or NTSR1 due to the expressed protein in the biological sample. Furthermore, the NMU protein is known to have a cell proliferating activity. Therefore, the expression level of the NMU gene can be determined using such cell proliferating activity as an index. For example, cells which express GHSR1b and NTSR1 are prepared and cultured in the presence of a biological sample, and then by detecting the speed of proliferation, or by measuring the cell cycle or the colony forming ability the cell proliferating activity of the biological sample can be determined.

Moreover, in addition to the expression level of the NMU gene, the expression level of other lung cell-associated genes, for example, genes known to be differentially expressed in NSCLC, may also be determined to improve the accuracy of the assessment. Such other lung cell-associated genes include those described in WO 2004/031413 and WO 2005/090603.

The patient to be assessed for the prognosis of NSCLC according to the method is preferably a mammal and includes human, non-human primate, mouse, rat, dog, cat, horse, and cow.

### III. A Kit for Assessing the Prognosis of NSCLC

The present invention provides a kit for assessing or determining the prognosis of NSCLC. Specifically, the kit comprises at least one reagent for detecting the expression of the NMU gene in a patient-derived biological sample, which reagent may be selected from the group of:
(a) a reagent for detecting mRNA of the NMU gene;
(b) a reagent for detecting the NMU protein; and
(c) a reagent for detecting the biological activity of the NMU protein.

Suitable reagents for detecting mRNA of the NMU gene include nucleic acids that specifically bind to or identify the NMU mRNA, such as oligonucleotides which have a complementary sequence to a part of the NMU mRNA. These kinds of oligonucleotides are exemplified by primers and probes that are specific to the NMU mRNA. These kinds of oligonucleotides may be prepared based on methods well known in the art. If needed, the reagent for detecting the NMU mRNA may be immobilized on a solid matrix. Moreover, more than one reagent for detecting the NMU mRNA may be included in the kit.

On the other hand, suitable reagents for detecting the NMU protein include antibodies to the NMU protein. The antibody may be monoclonal or polyclonal. Furthermore, any fragment or modification (e.g., chimeric antibody, scFv, Fab, F(ab′)₂, Fv, etc.) of the antibody may be used as the reagent, so long as the fragment retains the binding ability to the NMU protein. Methods to prepare these kinds of antibodies for the detection of proteins are well known in the art, and any method may be employed in the present invention to prepare such antibodies and equivalents thereof. Furthermore, the antibody may be labeled with signal generating molecules via direct linkage or an indirect labeling technique. Labels and methods for labeling antibodies and detecting the binding of antibodies to their targets are well known in the art and any labels and methods may be employed for the present invention. Moreover, more than one reagent for detecting the NMU protein may be included in the kit.

Furthermore, suitable reagents for detecting the biological activity or the NMU protein include, GHSR1b, NTSR1 or a heterodimer complex of the two proteins, and cells which express GHSR1b and NTSR1. The biological activity of the NMU protein can be detected, for example, by measuring the binding ability to GHSR1b or NTSR1 due to the expressed NMU protein in the biological sample. Alternatively, when a cell expressing GHSR1b and NTSR1 is used as the reagent, the biological activity can be determined by, for example, measuring the cell proliferating activity due to the expressed NMU protein in the biological. For example, the cell is cultured in the presence of a patient-derived biological sample, and then by detecting the speed of proliferation, or by measuring the cell cycle or the colony forming ability the cell proliferating activity of the biological sample can be determined. If needed, the reagent for detecting the NMU mRNA may be immobilized on a solid matrix. Moreover, more than one reagent for detecting the biological activity of the NMU protein may be included in the kit.

The kit may comprise more than one of the aforementioned reagents. Furthermore, the kit may comprise a solid matrix and reagent for binding a probe against the NMU gene or antibody against the NMU protein, a medium and container for culturing cells, positive and negative control reagents, and a secondary antibody for detecting an antibody against the NMU protein. For example, tissue samples obtained from patient with good prognosis or poor prognosis may serve as useful control reagents. A kit of the present invention may further include other materials desirable from a commercial and user standpoint, including buffers, diluents, filters, needles, syringes, and package inserts (e.g., written, tape, CD-ROM, etc.) with instructions for use. These reagents and such may be comprised in a container with a label. Suitable containers include bottles, vials, and test tubes. The containers may be formed from a variety of materials, such as glass or plastic.

As an embodiment of the present invention, when the reagent is a probe against the NMU mRNA, the reagent may be immobilized on a solid matrix, such as a porous strip, to form at least one detection site. The measurement or detection region of the porous strip may include a plurality of sites, each containing a nucleic acid (probe). A test strip may also contain sites for negative and/or positive controls. Alternatively, control sites may be located on a strip separated from the test strip. Optionally, the different detection sites may contain different amounts of immobilized nucleic acids, i.e., a higher amount in the first detection site and lesser amounts in subsequent sites. Upon the addition of test sample, the number of sites displaying a detectable signal provides a quantitative indication of the amount of NMU mRNA present in the sample. The detection sites may be configured in any suitably detectable shape and are typically in the shape of a bar or dot spanning the width of a test strip.

IV. Identifying Compounds that Inhibit the NMU Signaling Pathway

According to the present invention, GHSR1b and NTSR1, which were respectively known to bind to the NMU protein, were found to form a heterodimer complex which as a whole functions as an NMU receptor. Furthermore, the NMU protein was strongly suggested to be an important growth factor that might be associated with cancer cell invasion, functioning through the binding to the newly discovered heterodimer complex of GHSR1b and NTSR1. Therefore, compounds that inhibit the signal transduction by the NMU protein and this heterodimer can be used to inhibit the growth-promoting pathway of NSCLC and serve as agents for treating or preventing lung cancers, in particular, NSCLC. Thus, the present invention provides a method for identifying a compound that inhibits the signal transduction by the NMU protein and the heterodimer consisting of GHSR1b and NTSR1. Specifically, the method comprises the steps of:
(1) contacting a heterodimer of GHSR1b and NTSR1 with the NMU protein in the existence of a test compound;
(2) detecting the signal transduction by the heterodimer and the NMU protein; and
(3) selecting the test compound that inhibits the signal transduction by the heterodimer and the NMU protein.

The amino acid sequence of the proteins used for the method, i.e., GHSR1b, NTSR1, and NMU, are show as SEQ ID NOs: 4, 6, and 2, respectively.

According to an aspect of the present invention, functional equivalents of the heterodimer and the NMU protein may be used as the respective proteins in the method. Herein, a “functional equivalent” of a protein is a polypeptide that has a biological activity equivalent to the protein. Namely, any polypeptide that retains the binding ability of the NMU protein to GTSR1b and NTSR1 may be used as a functional equivalent of the NMU protein in the present method. On the other hand, any polypeptide that retains the binding ability toward the NMU protein and the ability to form a heterodimer complex with GTSR1b may be used as a functional equivalent of NTSR1; and those retaining the binding ability toward the NMU protein and the heterodimer complex forming ability with NTSR1 as a functional equivalent of GTSR1b. Such functional equivalents include fragments comprising the binding site of each of these proteins. For example, an NMU protein fragment ‘NMU-25’ that was shown to bind to the heterodimer complex in the Example described below can be used as a functional equivalent of the NMU protein, but the present invention is not restricted thereto.

In addition, such functional equivalents include those wherein one or more amino acids are substituted, deleted, added, or inserted to the natural occurring amino acid sequence of the respective proteins. Alternatively, the polypeptide may be one that comprises an amino acid sequence having at least about 80% homology (also referred to as sequence identity) to the sequence of the respective proteins. In other embodiments, the polypeptide can be encoded by a polynucleotide that hybridizes under stringent conditions (as defined above) to the natural occurring nucleotide sequence of the respective protein-encoding genes.

Generally, it is known that modifications of one or more amino acid in a protein do not influence the function of the protein. One of skill in the art will recognize that individual additions, deletions, insertions, or substitutions to an amino acid sequence which alters a single amino acid or a small percentage of amino acids is a “conservative modification” wherein the alteration of a protein results in a protein with similar functions. Conservative substitution tables providing functionally similar amino acids are well known in the art. For example, the following eight groups each contain amino acids that are conservative substitutions for one another:
1) Alanine (A), Glycine (G);
2) Aspartic acid (d), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cystein (C), Methionine (M) (see, e.g., Creighton, Proteins (1984)).

Such conservatively modified polypeptides are included in the present proteins. However, proteins applicable for the method are not restricted thereto and may include non-conservative modifications so long as they retain the binding ability to each other and for proteins forming the heterodimer, their ability to form a heterodimer with each of the other proteins. Furthermore, the modified proteins do not exclude polymorphic variants, interspecies homologues, and alleles of these proteins.

In addition to the above-mentioned modification, the proteins may be further linked to other substances so long as the proteins retain their binding ability and/or the ability to form a heterodimer complex. Usable substances include: peptides, lipids, sugar and sugar chains, acetyl groups, natural and synthetic polymers, etc. These kinds of modifications may be performed to confer additional functions or to stabilize the proteins.

The proteins used for the present method may be obtained from nature as naturally occurring proteins via conventional purification methods or through chemical synthesis based on the selected amino acid sequence. For example, conventional peptide synthesis methods that can be adopted for the synthesis includes:
(i) Peptide Synthesis, Interscience, New York, 1966;
(ii) The Proteins, Vol. 2, Academic Press, New York, 1976;
(iii) Peptide Synthesis (in Japanese), Maruzen Co., 1975;
(iv) Basics and Experiment of Peptide Synthesis (in Japanese), Maruzen Co., 1985;
(v) Development of Pharmaceuticals (second volume) (in Japanese), Vol. 14 (peptide synthesis), Hirokawa, 1991;
(vi) WO99/67288; and
(vii) Barany G. & Merrifield R. B., Peptides Vol. 2, “Solid Phase Peptide Synthesis”, Academic Press, New York, 1980, 100-118.

Alternatively, the proteins may be obtained adopting any known genetic engineering methods for producing polypeptides (e.g., Morrison J. (1977) J. Bacteriology 132: 349-51; Clark-Curtiss & Curtiss (1983) Methods in Enzymology (eds. Wu et al.) 101: 347-62). For example, first, a suitable vector comprising a polynucleotide encoding the objective protein in an expressible form (e.g., downstream of a regulatory sequence comprising a promoter) is prepared, transformed into a suitable host cell, and then the host cell is cultured to produce the protein. The protein may also be produced in vitro adopting an in vitro translation system.

Herein, the signal transduction by the heterodimer and NMU can be detected as either the binding between the heterodimer and NMU or the heterodimer activation, which includes any change occurring after the binding of the heterodimer and NMU. Therefore, the inhibition of the signal transduction by a compound can be detected by either detecting, under the presence of the compound, the binding between the heterodimer and NMU or the heterodimer activation. As a method for identifying compounds that inhibit the binding of the present invention, many methods well known by one skilled in the art can be used. Such identification can be carried out as an in vitro assay system, for example, in a cellular system. More specifically, first, either the hetrodimer complex or the NMU protein is bound to a support, and the other protein is contacted together with a test compound thereto. Next, the mixture is incubated, washed and the other protein bound to the support is detected and/or measured.

Example of supports that may be used for binding the proteins include insoluble polysaccharides, such as agarose, cellulose and dextran; and synthetic resins, such as polyacrylamide, polystyrene and silicon; preferably commercially available beads and plates (e.g., multi-well plates, biosensor chip, etc.) prepared from the above materials may be used. When using beads, they may be filled into a column. Alternatively, the use of magnetic beads is also known in the art, and enables to readily isolate proteins bound on the beads via magnetism.

The binding of a protein to a support may be conducted according to routine methods, such as chemical bonding and physical adsorption. Alternatively, a protein may be bound to a support via antibodies specifically recognizing the protein. Moreover, binding of a protein to a support can also be conducted by means of interacting molecules, such as the combination of avidin and biotin.

The binding between proteins is carried out in buffer, for example, but are not limited to, phosphate buffer and Tris buffer, as long as the buffer does not inhibit the binding between the proteins.

In the present invention, a biosensor using the surface plasmon resonance phenomenon may be used as a mean for detecting or quantifying the bound protein. When such a biosensor is used, the interaction between the proteins can be observed real-time as a surface plasmon resonance signal, using only a minute amount of polypeptide and without labeling (for example, BIAcore, Pharmacia). Therefore, it is possible to evaluate the binding between the heterodimer complex and the NMU protein using a biosensor such as BIAcore.

Alternatively, either the heterodimer complex or the NMU protein may be labeled, and the label of the bound protein may be used to detect or measure the bound protein. Specifically, after pre-labeling one of the proteins, the labeled protein is contacted with the other protein in the presence of a test compound, and then bound proteins are detected or measured according to the label after washing.

Labeling substances such as radioisotope (e.g., ³H, ¹⁴C, ³²P, ³³P, ³⁵S, ¹²⁵I, ¹³¹I), enzymes (e.g., alkaline phosphatase, horseradish peroxidase, β-galactosidase, β-glucosidase), fluorescent substances (e.g., fluorescein isothiosyanete (FITC), rhodamine) and biotin/avidin, may be used for the labeling of a protein in the present method. When the protein is labeled with radioisotope, the detection or measurement can be carried out by liquid scintillation. Alternatively, proteins labeled with enzymes can be detected or measured by adding a substrate of the enzyme to detect the enzymatic change of the substrate, such as generation of color, with absorptiometer. Further, in case where a fluorescent substance is used as the label, the bound protein may be detected or measured using fluorophotometer.

Furthermore, the binding in the present identification method can be also detected or measured using an antibody against the heterodimer or the NMU protein. Herein, an antibody against the heterodimer may be prepared by using the heterodimer as an antigen. Alternatively, either of the proteins forming the heterodimer, i.e., GTSR1b or NTSR1, may be used as the antigen so long as the prepared antibody recognizes the heterodimer. For example, after contacting the NMU protein immobilized on a support with a test compound and the heterodimer, the mixture is incubated and washed, and detection or measurement can be conducted using an antibody against the heterodimer. Alternatively, the heterodimer may be immobilized on a support, and an antibody against the NMU protein may be used as the antibody.

In case of using an antibody in the present screening, the antibody is preferably labeled with one of the labeling substances mentioned above, and detected or measured based on the labeling substance. Alternatively, the antibody against the heterodimer or the NMU protein may be used as a primary antibody to be detected with a secondary antibody that is labeled with a labeling substance. Furthermore, the antibody bound to the protein in the screening of the present invention may be detected or measured using protein G or protein A column.

Alternatively, in another embodiment of the identification method of the present invention, a two-hybrid system utilizing cells may be used (“MATCHMAKER Two-Hybrid system”, “Mammalian MATCHMAKER Two-Hybrid Assay Kit”, “MATCHMAKER one-Hybrid system” (Clontech); “HybriZAP Two-Hybrid Vector System” (Stratagene); the references “Dalton and Treisman, Cell (1992) 68: 597-612”, “Fields and Sternglanz, Trends Genet (1994)10: 286-92”). In the two-hybrid system, for example, the NMU protein is fused to the SRF-binding region or GAL4-binding region and expressed in yeast cells. The heterodimer complex that binds to the NMU protein is fused to the VP16 or GAL4 transcriptional activation region and also expressed in the yeast cells in the existence of a test compound. Alternatively, the heterodimer may be fused to the SRF-binding region or GAL4-binding region, and NMU to the VP16 or GAL4 transcriptional activation region. When the test compound does not inhibit the binding between the heterodimer complex and the NMU protein, the binding of the two activates a reporter gene, making positive clones detectable. As a reporter gene, for example, Ade2 gene, lacZ gene, CAT gene, luciferase gene and such can be used besides HIS3 gene.

In an embodiment of the present invention, the heterodimer complex consisting of GHSR1b and NTSR1 may be expressed on the surface of a living cell. Since these proteins are naturally expressed on the cell surface, it is possible to use cells, such as A549 and LC319, for the screening. Alternatively, using an expression vector(s), genes encoding these proteins may be introduced to suitable cells (e.g., COS-7) to express these proteins on the surface of the cells. The screening using cells that express the heterodimer on the cell surface permits not only identification of compounds that reacts with the heterodimer but characterization of the compounds that modulate receptor activity in the cell environment, which is a beneficial aspect for screening candidate compounds for pharmaceutical purposes.

When the proteins are expressed on the surface of a living cell, the signal transduction by the heterodimer and NMU can be detected by methods detecting the autocrine and paracrine signaling leading to stimulation of tumor cell growth (Heasley, Oncogene (2001) 20: 1563-9). For example, the inhibition of the signal transduction by a compound can be detected by:
(1) detecting the change in subcellular localization of the polypeptide including the ligand-induced internalization (Vandenbulcke F, et al. (2000) J. Cell Sci. 113: 2963-75; Lenkei Z, et al. (2000) J. Histochem. Cytochem. 48: 1553-63; Camina J P, et al. (2004) Endocrinology;145:930-40. Epub: (2003) as doi: 10.1210/en. 2003-0974; e.g., using a fluorescent labeled receptor protein; for example, labeled with CyHer5E receptor internalization assay fluorochrome (GE Healthcare));
(2) detecting the change in the concentration of cAMP in the cell (e.g., assays using FDSS (Fuctional Drug Screening System (Hamamatsu Photonics)) or FLIPR (Fluorometric Imaging Plate Reader (Molecular Devices)); Kojima M, et al. (2000) Biochem. Biophys. Res. Commun. 276: 435-8; Howard A D, et al. (2000) Nature 406: 70-4; Fujii R, et al. (2000) J. Biol. Chem. 275:21068-74; and methods using fluorescent labeled probes wherein the changes in fluorescence are detected (Pozzan T. et al. (2003) Eur. J. Biochem. 270: 2343-52));
(3) detecting the change in the activation of adenylate cyclase;
(4) detecting the change in the activation of protein kinase A (PKA);
(5) detecting the change in the expression of NMU target genes including FOXM1, GCDH, CDK5RAP1, LOC134145, and NUP188;
(6) detecting the change in cell proliferation, transformation, or any other oncogenic phenotype of the cell;
(7) detecting the change in the state of apoptosis of the cell;
(8) detecting the change in the interactivation between the heterodimer and G-protein (e.g., assays using FLIPR; Kojima M, et al. (2000) Biochem. Biophys. Res. Commun. 276: 435-8; Howard A D, et al. (2000) Nature 406: 70-4; Fuj ii R, et al. (2000) J. Biol. Chem. 275:21068-74);
(9) detecting the change in the activation of phospholipase C or its downstream pathway (Heasley L E. (2001) Oncogene 20: 1563-9);
(10) detecting the change in the activation of protein kinase cascade leading to activation of several kinases including Raf, MEK, ERKs, and protein kinase D (PKD) (Heasley L E. (2001) Oncogene 20: 1563 -9);
(11) detecting the change in the activation of a member of Src/Tec/Bmx-family of tyrosine kinases;
(12) detecting the change in the activation of a member of the Ras and Rho family, regulation of a member of the JNK members of MAP families, or the reorganization of the actin cytoskeleton (Heasley L E. (2001) Oncogene 20: 1563-9); and
(13) detecting the change in the activation of any signal complex mediated by the heterodimer activation.

Further, high throughput screening (HTS) can be conducted to identify compounds that inhibit the NMU signaling pathway. For example, HTS for such compounds can be performed through methods similar to those that identify compounds targeting G protein coupled receptors (Eglen R. M. (2005) Frontiers is Drug Design & Discovery 1: 97-111). Specifically, for HTS at the heterodimer of GHSR1b and NTSR1, the signal intensity changes can be measured (i) by reporter gene assays using expression systems engineered with cis-acting enhancer elements, DNA sequence motifs targeted by binding partners promoting gene expression (e.g., promoters of adenylate cyclase, FOXM1, GCDH, CDK5RAP1, LOC134145, NUP188, phospholipase C, Raf, MEK, ERKs, PKD, etc.) and upstream of a reporter gene; (ii) by second messenger assays (e.g., cAMP as the second messenger (Pozzan T. et al. (2003) Eur. J. Biochem. 270: 2343-52)); or (iii) as the accumulation of cAMP, inositol phospholipids, and such. However, different to the screening of compounds targeting G protein, Ca²⁺ cannot be used as an index for the change in the signal transduction of the NMU signaling pathway, since the treatment with NMU on cells expressing GHSR1b/NTSR1 did not change the intracellular Ca²⁺ level.

In general, such measurement on the signal intensity changes is conducted using a microtiter plate format (e.g., FLIPR, FDSS, etc.). However, the present invention is not restricted thereto.

Alternatively, the cellular protein redistribution can be measured to identify compounds that inhibit the signal transduction by the heterodimer and NMU by HTS via imaging-based analysis systems. It is known that the activation of the heterodimer via the binding of NMU to the receptor (heterodimer) causes redistribution of the receptor. For example, the redistribution of the heterodimer can be detected by examining fixed cells (cells treated or incubated with a test compound is compared to cells without a treatment or incubation with the test compound) via immunostaining techniques using antibodies recognizing either the native heterodimer or an epitope tag fused to the heterodimer. Alternatively, the measurement on the translocation of the heterodimer and NMU can be performed employing clonal cells that express labeled heterodimers, for example, those labeled with a suitable fluorescent protein (e.g., CypHer5, GFP) and detecting the redistribution of the label, which enables monitoring by automated confocal systems and analysis by imaging algorithms.

Any test compound, for example, cell extracts, cell culture supernatant, products of fermenting microorganism, extracts from marine organism, plant extracts, purified or crude proteins, peptides, non-peptide compounds, synthetic micromolecular compounds and natural compounds can be used in the screening methods of the present invention. The test compound of the present invention can be also obtained using any of the numerous approaches in combinatorial library methods known in the art, including (1) biological libraries, (2) spatially addressable parallel solid phase or solution phase libraries, (3) synthetic library methods requiring deconvolution, (4) the “one-bead one-compound” library method and (5) synthetic library methods using affinity chromatography selection. The biological library methods using affinity chromatography selection is limited to peptide libraries, while the other four approaches are applicable to peptide, non-peptide oligomer or small molecule libraries of compounds (Lam (1997) Anticancer Drug Des. 12: 145-67). Examples of methods for the synthesis of molecular libraries can be found in the art (DeWitt et al. (1993) Proc. Natl. Acad. Sci. USA 90: 6909-13; Erb et al. (1994) Proc. Natl. Acad. Sci. USA 91: 11422-6; Zuckermann et al. (1994) J. Med. Chem. 37: 2678-85; Cho et al. (1993) Science 261: 1303-5; Carell et al. (1994) Angew. Chem. Int. Ed. Engl. 33: 2059; Carell et al. (1994) Angew. Chem. Int. Ed. Engl. 33: 2061; Gallop et al. (1994) J. Med. Chem. 37: 1233-51). Libraries of compounds may be presented in solution (see Houghten (1992) Bio/Techniques 13: 412-21) or on beads (Lam (1991) Nature 354: 82-4), chips (Fodor (1993) Nature 364: 555-6), bacteria (U.S. Pat. No. 5,223,409), spores (U.S. Pat. No. 5,571,698; 5,403,484, and 5,223,409), plasmids (Cull et al. (1992), Proc. Natl. Acad. Sci. USA 89: 1865-9) or phage (Scott and Smith (1990) Science 249: 386-90; Devlin (1990) Science 249: 404-6; Cwirla et al. (1990) Proc. Natl. Acad. Sci. USA 87: 6378-82; Felici (1991) J. Mol. Biol. 222: 301-10; US Pat. Application 2002103360). The test compound exposed to a cell or protein according to the identification method of the present invention may be a single compound or a combination of compounds. When a combination of compounds is used in the method, the compounds may be contacted sequentially or simultaneously.

A compound isolated by the identification method of the present invention is a compound that inhibits the interaction of the heterodimer consisting of GHSR1b and NTSR1 with the NMU protein, and thus, is a candidate agent for treating or preventing diseases attributed to, for example, cell proliferative diseases, such as NSCLC. A compound in which a part of the structure of the compound obtained by the present method is converted by addition, deletion and/or replacement, is included in the compounds obtained by the identification methods of the present invention. A compound effective in suppressing the function of over-expressed genes, i.e., GHSR1b, NTSR1 or NMU gene, is deemed to have a clinical benefit and can be further tested for its ability to prevent cancer cell growth in animal models or test subjects.

### V. Screening Compounds for Treating or Preventing NSCLC

As explained above under the item of “IV. Identifying compounds that inhibit the NMU signaling pathway”, compounds that inhibit the signal transduction by the NMU protein and the heterodimer consisting of GHSR1b and NTSR1 may inhibit the growth-promoting pathway of NSCLC and may serve as an agent for treating or preventing lung cancers. Thus, the present invention provides a method of screening for a compound that can be used for treating or preventing NSCLC by identifying compounds that inhibit the signal transduction by the NMU protein and the heterodimer as detailed above.

If needed, compounds identified or screened through the present methods can be formulated into pharmaceutical compositions comprising the compounds as active ingredients. For the treatment and/or prevention of disorders, the compounds may be directly administered as a pharmaceutical composition to the patient or may be formulated according to conventional formulation methods. For example, if needed, the present polypeptides may be formulated into a form suitable for oral, rectal, nasal, topical (including buccal and sub-lingual), vaginal or parenteral (including intramuscular, sub-cutaneous, intravenous, intratumoral) administration, or for administration by inhalation or insufflation. Thus, the present invention encompasses pharmaceutical compositions which include any pharmaceutically acceptable excipient or carrier in addition to the compounds. The phrase “pharmaceutically acceptable” indicates that the substance is inert and includes conventional substances used as diluent or vehicle for a drug. Suitable excipients and their formulations are described, for example, in Remington's Pharmaceutical Sciences, 16^{th} ed. (1980) Mack Publishing Co., ed. Oslo et al.

Such pharmaceutical compositions may be used for treating and/or preventing disorders in human and any other mammal including mouse, rat, guinea-pig, rabbit, cat, dog, sheep, goat, pig, cattle, horse, monkey, baboon, and chimpanzee, particularly a commercially important animal or a domesticated animal.

The pharmaceutical compositions comprise the active ingredients (a polypeptide or polynucleotide of the present invention) at a pharmaceutically effective amount. A “pharmaceutically effective amount” of a compound is a quantity that is sufficient to treat and/or prevent disorders wherein the binding of the heterodimer complex and the NMU protein plays important roles. An example of a pharmaceutically effective amount may an amount that is needed to decrease the interaction between the heterodimer and NMU when administered to a patient, so as to thereby treat or prevent the disorders. The decrease in interaction may be, for example, at least a decrease of about 5%, 10%, 20%, 30%, 40%, 50%, 75%, 80%, 90%, 95%, 99%, or 100%. Alternatively, a pharmaceutically effective amount may be an amount that leads to a decrease in size, prevalence, or metastatic potential of NSCLC in a subject. Furthermore, when the pharmaceutical composition is applied prophylactically, the “pharmaceutically effective amount” may be an amount which retards or prevents occurrence of NSCLC or alleviates a clinical symptom of NSCLC.

The assessment of NSCLC to determine such a pharmaceutically effective amount of a compound identified through the present method can be made using standard clinical protocols including histopathologic diagnosis or through identification of symptomatic anomalies such as chronic cough, hoarseness, coughing up blood, weight loss, loss of appetite, shortness of breath, wheezing, repeated bouts of bronchitis or pneumonia, and chest pain.

The dose employed will depend upon a number of factors, including the age and sex of the subject, the precise disorder being treated, and its severity. Also the route of administration may vary depending upon the condition and its severity. However, the determination of an effective dose range for the identified compounds is well within the capability of those skilled in the art, especially in light of the detailed disclosure provide herein. The pharmaceutically or preventively effective amount (dose) of a compound can be estimated initially from cell culture assays and/or animal models.

If needed, a pharmaceutical composition comprising the identified compound may include any other therapeutic substance as an active ingredient so long as the substance does not inhibit the in vivo inhibiting effect of the compound. It should be understood that in addition to the ingredients particularly mentioned above, the formulations may include other agents conventional in the art having regard to the type of formulation in question.

In one embodiment of the present invention, a pharmaceutical composition comprising the identified compound may be included in articles of manufacture and kits containing materials useful for treating the pathological conditions of cancer, particularly NSCLC. The article of manufacture may comprise a container of any of the compounds with a label. Suitable containers include bottles, vials, and test tubes. The containers may be formed from a variety of materials, such as glass or plastic. The label on the container should indicate the composition is used for treating or preventing one or more conditions of the disease. The label may also indicate directions for administration and so on.

In addition to the container described above, a kit comprising a pharmaceutical composition comprising the identified compound may optionally comprise a second container housing a pharmaceutically-acceptable diluent. It may further include other materials desirable from a commercial and user standpoint, including other buffers, diluents, filters, needles, syringes, and package inserts with instructions for use.

The pharmaceutical compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration.

Hereinafter, the present invention is described in detail with reference to the Example. However, materials, methods and such described therein only illustrate aspects of the invention and in no way are intended to limit the scope of the present invention. As such, materials, methods and such similar or equivalent to those described therein may be used in the practice or testing of the present invention.

### Example

### 1. Experimental Procedures

### (1) Cell Lines and Clinical Tissue Samples

The human lung cancer cell lines used herein were as follows: 15 NSCLCs (A549, NCI-H23, NCI-H358, NCI-H522, NCI-H1435, NCI-H1793, LC174, LC176, LC319, PC3, PC9, PC14, SK-LU-1, RERF-LC-AI, and SK-MES-1); and 4 SCLCs (SBC-3, SBC-5, DMS114, and DMS273). All cells were grown in appropriate medium supplemented with 10% fetal calf serum (FCS) and were maintained at 37° C. in an atmosphere of humidified air with 5% CO₂.

37 primary NSCLC samples had been obtained earlier with informed consent from 37 patients (Kikuchi et al. (2003) Oncogene 22: 2192-205). Fifteen additional primary NSCLCs, seven ADCs and eight SCCs, were obtained along with adjacent normal lung tissue samples from patients who underwent surgery.

A total of 326 formalin-fixed primary NSCLCs (stages I-IIIa), more specifically, 224 ADCs, 86 SCCs, 13 large cell carcinomas (LCCs), and 3 adenosquamous carcinomas (ASCs), and adjacent normal lung tissue samples were obtained from patients. Advanced SCLC samples (stage IV) from post-mortem materials (17 individuals) obtained form patients were also used in this study. The use of all clinical materials was approved by the Institutional Research Ethics Committees.

### (2) Semiquantitative RT-PCR Analysis

Total RNA was extracted from cultured cells and clinical tissues using Trizol reagent (Life Technologies, Inc.) according to the manufacturer's protocol. Extracted RNAs and normal human tissue poly(A) RNAs were treated with DNaseI (Nippon Gene) and were reverse-transcribed using oligo(dT)₂₀ primer and SuperSpcript II reverse transcriptase (Invitrogen). Semiquantitative RT-PCR experiments were carried out with the following synthesized gene-specific primers and beta-actin (ACTB)-specific primers as internal control:

NMU,

(SEQ ID NO: 7)

5′-TGAAGAGATTCAGAGTGGACGA-3′
and

(SEQ ID NO: 8)

5′-ACTGAGAACATTGACAACACAGG-3′;
NMUIR,

(SEQ ID NO: 9)

5′-AAGAGGGACAGGGACAAGTAGT-3′
and

(SEQ ID NO: 10)

5′-ATGCCACTGTTACTGCTTCAG-3′;
NMU2R,

(SEQ ID NO: 11)

5′-GGCTCTTACAACTCATGTACCCA-3′
and

(SEQ ID NO: 12)

5′-TGATACAGAGACATGAAGTGAGCA-3′;
GHSR1a,

(SEQ ID NO: 13)

5′-TGGTGTTTGCCTTCATCCT-3′
and

(SEQ ID NO: 14)

5′-GAATCCCAGAAGTCTGAACA-3′;
GHSR1b,

(SEQ ID NO: 15)

5′-CTTGGGACACCAACGAGTG-3′
and

(SEQ ID NO: 16)

5′-AGGACCCGCGAGAGAAAGC-3′;
NTSR1,

(SEQ ID NO: 17)

5′-GGTCTGTGGCTGTGACTGAA-3′
and

(SEQ ID NO: 18)

5′-GTTTGAGCTGTGAGGGCTGT-3′;
GHRL,

(SEQ ID NO: 19)

5′-TGAGCCCTGAACACCAGAGAG-3′
and

(SEQ ID NO: 20)

5′-AAAGCCAGATGAGCGCTTCTA-3′;
NTS,

(SEQ ID NO: 21)

5′-TCTTCAGCATGATGTGTTGTGT-3′
and

(SEQ ID NO: 22)

5′-TGAGAGATTCATGAGGAAGTCTTG-3′;
FOXM1,

(SEQ ID NO: 23)

5′-CCCTGACAACATCAACTGGTC-3′
and

(SEQ ID NO: 24)

5′-GTCCACCTTCGCTTTTATTGAGT-3′;
unannotated transcript
(clone IMAGE: 3839141, mRA),

(SEQ ID NO: 25)

5′-AAAAAGGGGATGCCTAGAACTC-3′
and

(SEQ ID NO: 26)

5′-CTTTCAGCACGTCAAGGACAT-3′;
GCDH,

(SEQ ID NO: 27)

5′-ACACCTACGAAGGTACACATGAC-3′
and

(SEQ ID NO: 28)

5′-GCTATTTCAGGGTAAATGGAGTC-3′;
CDK5RAP1,

(SEQ ID NO: 29)

5′-CAGAGATGGAGGATGTCAATAAC-3′
and

(SEQ ID NO: 30)

5′-CATAGCAGCTTTAAAGAGACACG-3′;
LOC134145,

(SEQ ID NO: 31)

5′-CCACCATAACAGTGGAGTGGG-3′
and

(SEQ ID NO: 32)

5′-CAGTTACAGGTGTATGACTGGGAG-3′;
NUP188,

(SEQ ID NO: 33)

5′-CTGAATACAACTTCCTGTTTGCC-3′
and

(SEQ ID NO: 34)

5′-GACCACAGAATTACCAAAACTGC-3′;
CCNA2,

(SEQ ID NO: 35)

5′-AAATAGAGCGTGAAGATGCCCT-3′
and

(SEQ ID NO: 36)

5′-GGCAGCTGGCATCATTAATACTT-3′;
CCNB1,

(SEQ ID NO: 37)

5′-GGGTTCTTGTTTTATATACCTGGC-3′
and

(SEQ ID NO: 38)

5′-GAATTATGGCAGCAATCACAAG-3′;
CCND1,

(SEQ ID NO: 39)

5′-CTGGATGTTGTGTGTATCGAGAG-3′
and

(SEQ ID NO: 40)

5′-GTCTTCTGCTGGAAACATGCCG-3′;
and
ACTB,

(SEQ ID NO: 41)

5′-GAGGTGATAGCATTGCTTTCG-3′
and

(SEQ ID NO: 42)

5′-CAAGTCAGTGTACAGGTAAGC-3′.

PCR reactions were optimized for the number of cycles to ensure product intensity within the logarithmic phase of amplification.

### (3) Northern Blot Analysis

Human multiple-tissue blots (BD Biosciences Clontech) were hybridized with a ³²P-labeled PCR product of NMU. The full-length cDNA of NMU was prepared by RT-PCR using primers:

(SEQ ID NO: 43)

5′-CGCGGATCCGCGATGCTGCGAACAGAGAGCTG-3′
and

(SEQ ID NO: 44)

5′-CCGCTCGAGCGGAATGAACCCTGCTGACCTTC-3′.

Prehybridization, hybridization, and washing were performed according to the supplier's recommendations. The blots were autoradiographed with intensifying screens at room temperature for 72 hours.

### (4) Western Blotting

Cells were lysed with RIPA buffer (50 mM Tris-HCl (pH 8.0), 150 mM NaCl, 1% NP-40, 0.5% Na-deoxycholate, 0.1% SDS) containing Protease Inhibitor Cocktail Set III (CALBIOCHEM). Protein samples were separated by SDS-polyacrylamide gels and electroblotted onto Hybond-ECL nitrocellulose membranes (GE Healthcare Bio-sciences). Blots were incubated with rabbit polyclonal anti-NMU antibody (Alpha Diagnostic International), mouse monoclonal anti-FLAG M2 antibody (Sigma-Aldrich Co.), goat polyclonal anti-NTSR1 antibody (Santa Cruz Biotechnology, Inc.), and rabbit polyclonal anti-GHSR antibody (originally generated against peptide GVEHENGTDPWDTNEC (SEQ ID NO: 60)). Antigen-antibody complexes were detected using secondary antibodies conjugated to horseradish peroxidase (GE Healthcare Bio-sciences). Protein bands were visualized by ECL Western Blotting Detection Reagents (GE Healthcare Bio-sciences).

### (5) Immunohistochemistry and Tissue Microarray

To investigate the presence of NMU, GHSR1b, or NTSR1 protein in clinical samples (normal lung tissues, NSCLCs, and SCLCs embedded in paraffin blocks, respectively), the sections were stained with ENVISION+Kit/horseradish peroxidase (HRP) (DakoCytomation). Specifically, either a polyclonal antibody against NMU (Alpha Diagnostic International), GHSR1b (originally generated against peptide GGSQRALRLSLAGPILSLC (SEQ ID NO: 45)), or NTSR1 (Santa Cruz Biotechnology, Inc.) was added after blocking endogenous peroxidase and proteins, and the sections were incubated with HRP-labeled anti-rabbit IgG and anti-goat IgG as the secondary antibodies. Substrate-chromogen was added and the specimens were counterstained with hematoxylin.

Tumor tissue microarrays were constructed as previously published (Kononen et al. (1998) Nat. Med. 4: 844-7; Chin et al. (2003) Mol. Pathol. 56: 275-9; Callagy et al. (2003) Diagn. Mol. Pathol. 12: 27-34; Callagy et al. (2005) J. Pathol. 205: 388-96). The tissue area for sampling was selected based on a visual alignment with the corresponding HE-stained section on a slide. Three, four, or five tissue cores (diameter 0.6 mm; height 3-4 mm) taken from the donor tumor blocks were placed into a recipient paraffin block using a tissue microarrayer (Beecher Instruments). A core of normal tissue was punched from each case. 5-μm sections of the resulting microarray block were used for immunohistochemical analysis. NMU, GHSR1b, or NTSR1 positivity was assessed according to staining intensity as absent or positive by three independent investigators without prior knowledge of the clinical follow-up data. Cases were accepted only as positive if reviewers independently defined them as such.

### (6) Statistical Analysis

Tumor-specific survival curves were calculated from the date of surgery to the time of death related to NSCLC, or to the last follow-up observation. Kaplan-Meier curves were calculated for each relevant variable; differences in survival times among patient subgroups were analyzed using the Log-rank test.

### (7) Immunocytochemical Analyses

Cultured cells were washed twice with PBS(−), fixed in 4% paraformaldehyde solution for 60 min at room temperature, and rendered permeable with PBS(−) containing 0.1% Triton X-100 for 1.5 min. Prior to the primary antibody reaction, cells were covered with blocking solution (3% BSA in PBS(−)) for 60 min to block non-specific antibody binding. Then, the cells were incubated with antibodies to human NMU protein. Antibodies were stained with goat anti-rabbit secondary antibody conjugated to rhodamine (Cappel) for revealing endogenous NMU, and viewed with a microscope (DP50; OLYMPUS).

### (8) RNA Interference Assay

A vector-based RNA interference (RNAi) system, psiH1BX3.0, had been previously established by the present inventors to direct siRNA synthesis in mammalian cells (Suzuki et al. (2003) Cancer Res. 63: 7038-41; Suzuki et al. (2005) Cancer Res. 65: 11314-25; Kato et al. (2005) Cancer Res. 65: 5638-46; Furukawa et al. (2005) Cancer Res. 65: 7102-10). 10 μg siRNA-expression vector was transfected using 30 μl of Lipofectamine 2000 (Invitrogen) into NSCLC cell lines A549 and LC319, both of which endogenously overexpress NMU, GHSR1b, NTSR1, and FOXM1. The transfected cells were cultured for five days in the presence of appropriate concentrations of Geneticin (G418), and afterwards, the cell numbers and viability were measured by Giemsa staining and triplicate MTT assays. The target sequences of the synthetic oligonucleotides for RNAi were as follows:

control 1 (EGFP: enhanced green fluorescent
protein (GFP) gene, a mutant of Aequorea
victoria GFP),

(SEQ ID NO: 46)

5′-GAAGCAGCACGACTTCTTC-3′;
control 2 (Luciferase:
Photinus pyralis luciferase gene),

(SEQ ID NO: 47)

5′-CGTACGCGGAATACTTCGA-3′;
control 3 (Scramble: chloroplast Euglena gracilis
gene coding for 5S and 16S rRNAs),

(SEQ ID NO: 48)

5′-GCGCGCTTTGTAGGATTCG-3′;
siRNA-NMU (si-NMU),

(SEQ ID NO: 49)

5′-GAGATTCAGAGTGGACGAA-3′;
siRNA-GHSR-1 (si-GHSR-1),

(SEQ ID NO: 50)

5′-CCTCTACCTGTCCAGCATG-3′;
siRNA-GHSR-2 (si-GHSR-2),

(SEQ ID NO: 51)

5′-GCTGGTCATCTTCGTCATC-3′;
siRNA-NTSR1-1 (si-NTSR1-1),

(SEQ ID NO: 52)

5′-GTTCATCAGCGCCATCTGG-3′;
siRNA-NTSR1-2 (si-NTSR1-2),

(SEQ ID NO: 53)

5′-GGTCGTCATACAGGTCAAC-3′;
and
siRNA-FOXM1 (si-FOXM1),

(SEQ ID NO: 54)

5′-GCAGCAGAAACGACCGAAT-3′.

To validate the RNAi system, individual control siRNAs (EGFP, Luciferase, and Scramble) were initially confirmed using semiquantitative RT-PCR to decrease expression of the corresponding target genes that had been transiently transfected into COS-7 cells. Down-regulation of NMU, GHSR1, NTSR1, and FOXM1 expression by their respective siRNAs (si-NMU, si-GHSR1, si-NTSR1-1, si-NTSR1-2, and si-FOXM1), but not by controls, was confirmed with semiquantitative RT-PCR in the cell lines used for this assay.

### (9) Flow Cytometry

Cells were plated at a density of 5×10⁵ cells/100-mm dish, transfected with siRNA-expression vectors, and cultured in the presence of appropriate concentrations of geneticin. Four days after transfection, the culture medium was replaced with geneticin-free medium. Cells were incubated for additional 24 hours, then trypsinized, collected in PBS, and fixed in 70% cold ethanol for 30 min. After treatment with 100 μg/ml RNase (Sigma-Aldrich Co.), the cells were stained with 50 μg/ml propidium iodide (Sigma-Aldrich Co.) in PBS. Flow cytometry was performed on Becton Dickinson FACSCalibur and analyzed by Cell Quest software (Becton Dickinson Biosciences). The percentage of nuclei in G0/G1, S, and G2/M phases on the cell cycle and the sub-GI population, were determined from at least 20,000 ungated cells.

### (10) NMU-Expressing COS-7 Transfectants

NMU-expressing stable transfectants were established according to a standard protocol. The entire coding region of NMU was amplified by RT-PCR using the primer set described above. The product was digested with BamHI and XhoI, and cloned into appropriate sites of pcDNA3.1-myc/His A(+) vector (Invitrogen) that contains the c-myc-His-epitope sequence (LDEESILKQE-HHHHHH (SEQ ID NO: 55)) at the C-terminus of the NMU protein. Using FuGENE 6 Transfection Reagent (Roche Diagnostics) according to the manufacturer's instructions, COS-7 cells, avoid of endogenous NMU expression, were transfected with plasmids expressing either NMU (pcDNA3. 1 -NMU-myc/His), an antisense strand of NMU (pcDNA3.1-antisense), or mock (pcDNA3.1) plasmids. Transfected cells were cultured in DMEM containing 10% FCS and geneticin (0.4 mg/ml) for 14 days; then 50 individual colonies were trypsinized and screened for stable transfectants by limiting-dilution assay. Expression of NMU was determined in each clone by RT-PCR, Western blotting, and immunostaining.

(11) Cell-Growth and Colony-Formation Assays COS-7 transfectants that stably express NMU were seeded onto 6-well plates (5×10⁴ cells/well), and maintained in medium containing 10% FCS and 0.4 mg/ml geneticin for 24, 48, 72, 96, 120, and 144 hours. At each time point, cell proliferation was evaluated by MTT assay using Cell Counting Kit (WAKO). Colonies were counted at 144 hours. All experiments were done in triplicate. Interaction of NMU-25 with COS-7 cells were examined by flow-cytometric analysis. Specifically, subconfluent cells were harvested in Cell Dissociation Solution (Sigma-Aldrich Co.) and suspended in DMEM. Then, 1×10⁶ cells/microtube were washed with assay buffer (PBS(−) with 10 mM MgCl₂, 2 mM EDTA, and 0.1% BSA), and the cells were incubated with 0.5-10 μM rhodamine-labeled NMU-25 peptide (NMU-25-rhodamine; Phoenix Pharmaceuticals, Inc.) in assay buffer for 2 hours at room temperature. Subsequently, the cells were washed twice with assay buffer.

To detect the population of cells binding to rhodamine-labeled NMU-25, flow cytometry was performed using Becton Dickinson FACSCalibur and analyzed by Cell Quest software. The growth effect of NMU on NSCLC cells was also examined using LC319 cells transiently transfected with plasmids expressing NMU or mock plasmids. The cells were cultured in RPMI containing 10% FCS and geneticin (1 mg/ml) for 18 days, and colonies were counted.

### (12) Matrigel Invasion Assay

COS-7 cells transiently transfected with plasmids expressing NMU or mock plasmid were grown to nearly confluence in DMEM containing 10% fetal bovine serum. The cells were harvested by trypsinization and subsequently washed in DMEM without addition of serum or proteinase inhibitor. The cells were suspended in DMEM at 1×10⁵ cells/ml. Before preparing a cell suspension, a dried layer of Matrigel matrix (Becton Dickinson Labware) was rehydrated with DMEM for 2 hours at room temperature. DMEM (0.75 ml) containing 10% fetal bovine serum was added to each lower chamber of 24-well Matrigel invasion chambers, and 0.5 ml (5×10⁴ cells) of cell suspension was added to each insert of the upper chamber. The plates of inserts were incubated for 22 hours at 37° C. After the incubation, the chambers were processed and the cells invading through the Matrigel-coated inserts were fixed and stained by Giemsa as directed by the supplier (Becton Dickinson).

### (13) Autocrine Assay and Inhibition of Cell Growth by Anti-NMU Antibody

To confirm the autocrine function of NMU in the growth of mammalian cells, COS-7 cells were cultured in medium containing the active form of a 25-amino-acid polypeptide of NMU (NMU-25; Sigma-Aldrich Co.) at final concentrations of 0.3 to 15 μM. The same concentrations of BSA served as controls. The peptides/proteins were added every 48 hours for 6 days. At the 144-hours time point, cell proliferation was evaluated by MTT and colony-formation assays.

Next, anti-NMU antibody (rabbit polyclonal anti-NMU-25 antibody; Phoenix Pharmaceuticals, Inc.) was investigated whether it can neutralize the effect of NMU on cell growth by blocking the binding of NMU-25 to its receptors. COS-7 cells were cultured in media containing 3 μM NMU-25 and anti-NMU antibody at concentrations of 0.5 to 7.5 μM. To confirm the ability of anti-NMU antibody to inhibit the growth of NSCLC cells that endogenously express NMU, LC319 or A549 cells were cultured for 4 days in media containing anti-NMU antibody at concentrations of 0.5 to 7.5 μM. LC176 cells, which scarcely express NMU, were used under the same culture conditions as control of the assay. Cell viability was evaluated by MTT assay. Each experiment was done in triplicate.

### (14) Ligand-Receptor Binding Assay

To confirm the binding of NMU-25 to endogenous candidate receptors on the NSCLC cell, a receptor-ligand binding assay using LC319 and PC14 cells that express GHSR1b and NTSR1, but not NMU1R and NMU2R were performed. Specifically, trypsinized cells were seeded onto a 96-well (with black wall and clear bottom) microtiter plates 24 hours prior to the assay. The medium was removed and the cells were incubated with CyS-labeled NMU-25 peptide (1 μM) with or without the addition of 10-fold excess unlabeled NMU-25 peptide as the competitor. The plate was incubated in dark for 24 hours at 37° C. and then scanned on 8200 Cellular Detection System (Applied Biosystems) to quantify the amount of CyS fluorescence probe bound to the surface of each cell. (15) Immunocytochemistry for Internalized Receptors

To investigate the association of NMU-25 with its candidate receptors, GHSR1b and NTSR1, the following experiments were performed. The entire coding region of each of the receptor genes was amplified by RT-PCR using primers:

GHSR1b
(5′-GGAATTCCATGTGGAACGCGACGCCCAGCGAA-3′ (SEQ ID
NO: 56)
and
5′-CGCGGATCCGCGGAGAGAAGGGAGAAGGCACAGGGA-3′), (SEQ
ID NO: 57)
and
NTSR1
(5′-GGAATTCCATGCGCCTCAACAGCTCCGCGCCGGGAA-3′ (SEQ
ID NO: 58)
and
5′-CGCGGATCCGCGGTACAGCGTCTCGCGGGTGGCATTGCT-3′ (SEQ
ID NO: 59).

The products were digested with EcoRI and BamHI and cloned into appropriate sites of p3XFLAG-CMV10 vector (Sigma-Aldrich Co.). COS-7 cells were transfected with FLAG-tagged GHSR1b or NTSR1 expression plasmid using FuGENE 6 Transfection Reagent as described above. The cells subjected to internalization assays were exposed to NMU-25 (10 μM) for 120 min. The cells were then fixed with 4% paraformaldehyde solution for 15 min at 37° C., and washed with PBS(−). Specimens were incubated in PBS(−) containing 0.1% Triton X-100 for 10 min and subsequently washed with PBS(−). Prior to primary antibody reaction, the cells were incubated in CAS-BLOCK (ZYMED Laboratories Inc.) for 10 min to block non-specific antibody binding. Then, the cells were incubated with both rabbit polyclonal and anti-GHSR antibody and goat polyclonal anti-NTSR1 antibody. The antibodies were stained with both anti-rabbit secondary antibody conjugated to Alexa Fluor 488 (Molecular Probes) and anti-goat secondary antibody conjugated to Alexa Fluor 594 (Molecular Probes). DNA was stained with 4′,6-diamidino-2-pheylindole (DAPI). Images were viewed and assessed using confocal microscopy (TCS SP2 AOBS; Leica Microsystems).

(16) Internalization Study with Fluorescence Ligand of NMU

LC319 cells were grown in DMEM containing 10% FCS. The cells were washed in PBS(−), and preincubated for 10 min at 37° C. in DMEM containing 0.1% BSA. The cells were then incubated for various periods of time with Alexa Fluor 594-labeled NMU-25 peptide in DMEM containing 0.1% BSA. At the end of incubation, the cells were washed three times with ice-cold PBS(−), fixed with 4% paraformaldehyde solution, initially for 5 min on ice and then for 15 min at room temperature. The cells were washed, and treated with DAPI. Images were viewed and assessed using confocal microscopy (TCS SP2 AOBS; Leica Microsystems). Optical sections with intervals of 0.25 μm were taken with 63×/1.4 objective.

### (17) Detection of Receptor Dimerization

Cultured cells were washed twice with ice-cold PBS(−) and incubated with 5 mM Dithiobis [succinimidyl propionate] (DSP) (PIERCE) for 60 min in PBS(−) on ice. The reaction was quenched by incubation with Stop solution (1 M Tris, pH 7.5) at a final concentration of 50 mM Tris for 15 min on ice. The cells were then washed twice with ice-cold PBS(−) and lysed in ice-cold Tx/G buffer (300 mM NaCl, 1% Triton X-100, 10% glycerol, 1.5 MM MgCl₂, 1 mM CaCl₂, and 10 mM iodoacetamide in 50 mM Tris-Cl, pH 7.4) containing protease inhibitor (Protease Inhibitor Cocktail Set III; Calbiochem) for 60 min on ice. Iodoacetamide was included in each buffer used for protein preparation to prevent non-specific disulfide linkages. The lysates were then centrifugated for 15 min at 15,000 rpm at 4° C. and the supernatants were incubated with anti-FLAG M2-agarose affinity beads (Sigma-Aldrich Co.) at 4° C. overnight. The immunoprecipitates (containing cell surface receptors) were collected, washed three times with TBST buffer (150 mM NaCl, 0.05% Tween-20 in 20 mM Tris-Cl, pH 7.6), and eluted in no-reducing Laemmli sample buffer. The solutions were subjected to SDS-PAGE, and receptor proteins were detected by Western blot analysis using mouse monoclonal anti-FLAG M2 antibody, goat polyclonal anti-NTSR1 antibody, or rabbit polyclonal anti-GHSR antibody as the primary antibody, and rec-Protein G-Peroxidase Conjugate (ZYMED Laboratories, Inc.) to detect the antigen-antibody complexes.

(18) Measurement of cAMP Levels

Trypsinized LC319, REF-LC-AI, NCI-H358 and SK-MED-1 cells were seeded onto a 96-well microtiter plate (5.0×10⁴ cells) and cultured in appropriate medium supplemented with 10% FCS for 24 hours, and then the medium was changed to serum free/1 mM IBMX (isobutylmethylxanthine) 20 min prior to the assay. Next, the cells were incubated with individual concentrations of peptides (NMU-25, GHRL, or NTS) for 20 min and cAMP levels of the cells were measured using cAMP EIA System (GE Healthcare Bio-sciences).

### (19) Intracellular Ca²⁺ Mobilization Assay

Trypsinized LC319 cells were seeded onto poly-D-lysine coated 384-well black-wall, clear-bottom microtiter plate (1.0×10⁴ cells/ml) 24 hours prior to the assay. The cells were loaded for 1 hour with 4 μM Fluo-3-AM fluorescent indicator dye in assay buffer (Hank's balanced salt solution, 20 mM HEPES, 2.5 mM probenecid), washed three times with assay buffer, and then incubated for 10 min at room temperature before detection on fluorometric imaging plate reader (FLIPR; Molecular Devices). Fluorescence data of Ca²⁺ release were collected in real-time at 1 sec intervals for the first 65 sec and at 3 sec intervals for additional 300 sec after individual concentrations of peptide (NMU-25, GHSR, or NTS) treatment. Maximum change in fluorescence over baseline was measured to determine the response of the cells to the individual peptide stimulations.

(20) Identification of Downstream Genes of NMU by cDNA Microarray

LC319 cells were transfected with either siRNA against NMU (si-NMU or Luciferase (LUC; control siRNA). mRNAs were extracted 0, 6, 12, 24, 36, 48, and 60 hours after the transfection, labeled with Cy5 or Cy3 dye, and subjected to co-hybridization onto cDNA microarray slides containing 32,256 genes as described (Kakiuchi et al. (2003) Mol. Cancer Res. 1: 485-99; Kakiuchi et al. (2004) Hum. Mol. Genet. 13: 3029-43; Ochi et al. (2004) Int. J. Oncol. 24: 647-55). After normalization of the data, genes with signals higher than the cut-off value were further analyzed. Genes whose intensity was significantly decreased in accordance with the reduction of NMU expression were initially selected using SOM cluster analysis (Kohonen (1990) Proceedings of the IEEE 78: 1464-80). Validation of candidate downstream genes of NMU was performed using semiquantitative RT-PCR experiments of the same mRNAs from LC319 cells used for the microarray hybridization, with the gene-specific primers.

### 2. Results

### (1) NMU in Lung Tumors and Normal Tissues

To search for novel target molecules for the development of therapeutic agents and/or diagnostic markers for NSCLC, first, genes that showed 5-fold higher expression in more than 50% of 37 NSCLCs analyzed by cDNA microarray were screened. Among the 23,040 screened genes, NMU transcript was identified as being frequently overexpressed in the tested NSCLCs and increased NMU expression was also confirmed in the majority of additional tested NSCLC cases. In addition, up-regulation of NMU in 13 of the examined 15 NSCLC cell lines and in all of the examined 4 small-cell lung cancer (SCLC) cell lines was observed. Northern blotting with NMU cDNA as a probe identified a 0.8 kb transcript as a very weak band only in the brain and stomach among the examined 15 normal human tissues. NMU expression was also examined in clinical lung cancers using tissue microarray system. Positive staining of the cytoplasm was observed in 68% of surgically resected NSCLCs (220/326), and 82% of SCLCs (14/17), while no staining was observed in any of the examined normal lung tissues. NSCLC patients with NMU-positive tumors were found to exhibit shorter cancer-specific survival times than patients whose tumors were negative for NMU (p=0.036 by the Log-rank test) (FIG. 1).

### (2) Effect of NMU on the Growth of NSCLC Cells

To assess whether NMU is essential for the growth and survival of lung-cancer cells, a plasmid expressing siRNA against NMU (si-NMU) was designed and constructed, in addition to three different control plasmids (siRNAs for EGFP, Luciferase (LUC), and Scramble (SCR)). The si-NMU expressing plasmid was transfected into A549 and LC319 cells to suppress the expression of endogenous NMU. The amount of NMU transcript in the cells transfected with si-NMU was significantly decreased in comparison with cells transfected with any of the three control siRNAs. Furthermore, transfection of si-NMU also resulted in significant decrease in cell viability and colony numbers measured by MTT and colony formation assay, respectively. To further investigate the molecular mechanisms of this growth suppression, flow cytometry after transfection of si-NMU into NSCLC cells was performed to discover that the sub-GI fraction (45.6%) in LC319 cells transfected with si-NMU was significantly larger than in cells transfected with si-EGFP (11.2%).

### (3) Autocrine Growth-Promoting Effect of NMU

To disclose the potential role of NMU in tumorigenesis, plasmids designed to express either NMU (pcDNA3.1-NMU-myc/His) or a complementary strand of NMU (pcDNA3.1-antisense) were prepared. Each of these two plasmids were transfected into COS-7 cells and the expression of NMU protein was confirmed in the cytoplasm and Golgi structures by immunocytochemical staining using anti-NMU antibody.

To determine the effect of NMU on the growth or mammalian cells, colony-formation assay of COS-7-derived transfectants that stably express NMU was carried out. Immunocytochemical analysis using anti-NMU antibody detected NMU protein in more than 90% of the COS-7 cells in the culture. Three independent COS-7 cell lines expressing exogenous NMU (COS-7-NMU-1, -2, and -3) were established, and their growth was compared to the growth of control cells that were transfected with either the antisense strand or a mock vector (COS-7-AS-1, and -2; and COS-7-mock). The growth of all of the three COS-7-NMU cells was promoted at a significant degree in accordance with the expression level of NMU. There was also a remarkable tendency in COS-7-NMU cells to form larger colonies than the control cells. Furthermore, colony-formation assays were performed to investigate whether NMU can act as a growth promoting factor for lung-cancer cells (LC319). The number of geneticin-resistant colonies was significantly increased in dishes containing LC3 19 cells that had been transfected with the sense-strand of cDNA (pcDNA3.1 -NMU-myc/His) corresponding to the normal transcript in comparison to the cells transfected with the mock vector.

As the immunohistochemical analysis on tissue microarray had indicated that lung-cancer patients with NMU positive tumors showed shorter cancer-specific survival period than patients whose tumors were negative for NMU, Matrigel invasion assays using COS-7-NMU cells were performed. Invasion of COS-7-NMU cells through Matrigel was significantly enhanced compared to the control cells transfected with the mock plasmid, suggesting that NMU could also contribute to the highly malignant phenotype of lung-cancer cells.

Subsequently, autocrine assays were carried out using the commercially-available active NMU-25, a 25 amino acid polypeptide. To investigate whether NMU-25 would affect the cell growth, COS-7 cells were incubated with either NMU-25 or bovine serum albumin (BSA) (control) at final concentrations of 0.3 to 15 μM in the culture media. COS-7 cells incubated with NMU-25 showed enhanced cell growth in a dose-dependent manner by both the MTT and colony-formation assays, compared to the control. The flow cytometry detected that rhodamine-labeled NMU-25 peptide bound to the surface of COS-7 cells in a dose-dependent manner. The results suggest that the growth promoting effect of NMU was likely to be mediated through the binding of NMU-25 to a receptor(s) on the cell surface of COS-7. Subsequently, anti-NMU antibody (0.5 to 7.5 μM) was investigated whether it can inhibit the growth of COS-7 cells cultured in medium containing 3 μM of NMU-25. Expectedly, the growth enhancement caused by the addition of 3 μM of NMU-25 was neutralized by the addition of 7.5 μM anti-NMU antibody, and the viability of COS-7 cells became almost equivalent to that of cells cultured without NMU-25.

Then, the effect of anti-NMU antibody (0.5 to 7.5 μM) on the growth of two lung-cancer cell lines, LC319 and A549, which showed high levels of endogenous NMU expression, was examined. The growth of both cell lines was suppressed by the addition of anti-NMU antibody into their culture media, in a dose-dependent manner (p<0.0001, and p=0.0002, respectively; each paired t test), while that of LC176 cells expressing NMU at a hardly-detectable level was not affected. These data indicate that NMU functions as an autocrine/paracrine growth factor for the proliferation of NSCLC cells.

### (4) GHSR1/NTSR1 as Receptors for NMU in a Growth-Promoting Pathway

Two known NMU receptors, NMU1R (FM3/GPR66) and NMU2R (FM4), play important roles in energy homeostasis (Fujii et al. (2000) J. Biol. Chem. 275: 21068-74; Howard et al. (2000) Nature 406: 70-4; Funes et al. (2002) Peptides 23: 1607-15). NMU1R is present in many peripheral human tissues (Fuji et al. (2000) J. Biol. Chem. 275: 21068-74; Howard et al. (2000) Nature 406: 70-4; Funes et al. (2002) Peptides 23: 1607-15), but NMU2R is located only in the brain. To investigate whether NMU1R and NMU2R genes are expressed in NSCLCs and are responsible for the growth promoting effect, the expression of these NMU receptors were analyzed in normal human brain and lung, NSCLC cell lines, and in clinical tissues by semiquantitative RT-PCR experiments. Neither NMU1R nor NMU2R expression was detected in any of the cell lines or clinical samples examined, although NMU1R was expressed in lung and NMU2R in brain, suggesting that NMU is likely to mediate its growth-promoting effect through interaction with other receptor(s) in lung cancer cells.

NMU1R and NMU2R were originally isolated as homologues of known neuropeptide GPCRs. An unidentified NMU receptor(s) having some degree of homology to NMU1R and/or NMU2R was speculated to be involved in the signaling pathway. Therefore, BLAST program was used to search for candidate NMU receptors. The homology and expression patterns of genes in NSCLCs in the expression profile data of the present inventors picked up GHSR1b (GenBank Accession No. NM_{—}004122; SEQ ID NOs: 3 and 4) and NTSR1 (GenBank Accession No. NM_{—}002531; SEQ ID NOs: 5 and 6) as good candidates.

GHSR has two transcripts, type 1a and 1b. The human GHSR type 1a cDNA encodes a predicted polypeptide of 366 amino acids with seven transmembrane domains, a typical feature of a G protein-coupled receptor. A singly intron separates its open reading frame into two exons encoding the transmembrane domains 1-5 and 6-7, placing GHSR1a into the intron-containing class of GPCRs. Type 1b is a non-spliced mRNA variant transcribed from a single exon that encodes a polypeptide of 289 amino acids with five transmembrane domains.

According to semiquantitative RT-PCR analysis using specific primers for each of the variants, GHSR1a was indicated not to be expressed in NSCLCs. On the other hand, GHSR1b and NTSR1 were expressed at a relatively high level in some NSCLC cell lines, but not in normal lung. The GHSR1b product reveals 46% homology to NMU1R, and NTSR1 encodes 418 amino acids with 47% homology to NMU1R. COS-7 cells examined on autocrine growth-promoting effect of NMU as described above, were confirmed by semiquantitative RT-PCR analysis to endogenously express both GHSR1b and NTSR1. Further, immunohistochemical analysis with anti-GHSR1b and anti-NTSR1 polyclonal antibodies were performed using tissue microarrays consisting of 326 NSCLC tissues. Of the 326 cases, GHSR1b staining was positive for 218 (67%); and 217 cases were positive for NTSR1 (67%). The expression pattern of GHSR1b or NTSR1 was significantly concordant with the NMU expression in these tumors (Chi-square=68 and 79;p<0.0001 and <0.0001, respectively).

To investigate the binding of NMU-25 to the endogenous GHSR1b and NTSR1 on the NSCLC cells, receptor-ligand binding assay using LC319 and PC14 cells treated with NMU-25 (1 μM) was performed. Cy5-labeled NMU-25 was detected to bind to the surface of these two cells lines that endogenously express both of the two novel receptors (GHSR1b and NTSR1) but no detectable NMU1R and/or NMU2R. The binding activity was elevated in a dose-dependent manner and was inhibited by the addition of 10-fold excess unlabeled NMU-25 as a competitor, suggesting specific interaction of NMU-25 to these cells.

Biologically active ligands for GPCRs have been reported to specifically bind to their cognate receptors and cause an increase in second-messengers, such as intracellular Ca²⁺ and/or cyclic adenosine monophosphate (cAMP) levels. Therefore, the ability of NMU for the induction of these second-messengers was determined in LC319 cells through its interaction with GHSR1b/NTSR1. Enhancement of cAMP production, but not of Ca²+flux was detected by NMU-25 in a dose-dependent manner in LC319 cells that express both GHSR1b and NTSR1, when the cells were cultured in the presence of NMU-25 at final concentrations of 3 to 100 μM in the culture media.

The results demonstrated that NMU-25 activated the NMU-25-related signaling pathway possibly through functional GHSR1b/NTSR1 in NSCLC cells. This effect was likely to be NMU-25 specific, because the addition of the same amount of GHRL and NTS, known ligands for GHSR/NTSR1, did not enhance the cAMP production. On the other hand, the treatment with NTS, but not the treatment with GHRL caused the mobilization response of intracellular Ca²⁺ in LC319 cells as similar to the previous reports (Kojima et al. (1999) Nature 402: 656-60; Heasley et al. (2001) Oncogene 20: 1563-9; Petersenn et al. (2001) Endocrinology 142: 2649-59), suggesting the ligand-dependent and diverse physiologic function of GHSR1b and/or NTSR1 in mammalian cells.

Then, the biological significance of NMU-receptor interaction was examined in pulmonary carcinogenesis using plasmids designed to express siRNA against GHSR or NTSR1 (si-GHSR-1, si-NTSR1-1, and si-NTSR1-2). The transfection of either of these plasmids into A549 or LC319 cells suppressed the expression of the endogenous receptor in comparison to cells containing any of the three control siRNAs. In accordance with the reduced expression of the receptors, A549 and LC319 cells showed significant decreases in cell viability and numbers of colonies. These results strongly support the possibility that NMU, by interaction with GHSR1b and NTSR1, might play a very significant role in the development and/or progression of lung cancer.

(5) Internalization of GHSR1b/NTSR1 Receptors after Binding with NMU

To determine the mechanism involved in the regulation of NMU-GHSR1b/NTSR1 signaling, GHSR1b/NTSR1 was examined whether it is internalized when they are exposed to NMU, through confocal microscopy observation of the subcellular distribution of the two receptors after NMU-25 stimulation. After their introduction into COS-7 cells, the GHSR1b and NTSR1 receptors were mainly co-located at the plasma membrane under the condition without exposure to NMU-25. However, once NMU-25 was added to the cell culture, both of the two receptors were co-internalized and predominantly formed vesicle-like structure in a time-dependent manner. Similarly, in LC319 cells, which endogenously overexpress both GHSR1b and NTSR1, NMU-stimulation induced co-internalization of the two receptors. The results obtained by monitoring both the endogenously and exogenously expressed receptors, suggest the possibility of physical interaction between GHSR1b and NTSR1 as well as NMU-induced co-internalization.

To further confirm whether NMU is internalized after the binding to its receptors, internalization of NMU was investigated using Alexa Fluor 594-labeled NMU-25 (NMU-25 Alexa594) by confocal microscopy. The binding of agonists to GPCRs on the cell surface is generally known to initiate receptor mediated endocytosis. In the course of this process, receptors are passed through multiple intracellular pathways that lead to lysosomal degradation or recycling them to the cell surface (Bohm et al. (1997) Biochem. J. 322: 1-18; Koenig et al. (1997) Trends Pharmacol. Sci. 18: 276-87). On the other hand, far less is known about whether all GPCR-ligands are internalized together with their receptor. In the case of neuropeptides, the ligand is usually internalized with its receptor (Ghinea et al. (1992) J. Cell Biol. 118: 1347-58; Grady et al. (1995) Mol. Biol. Cell 6: 509-24; Vandenbulcke et al. (2000) J. Cell. Sci. 113: 2963-75). The xz- and yz-projections indicated that NMU-25-Alexa594 was incorporated within the cells. After the 15-min incubation, the internalized ligand was concentrated in dots or irregular clusters at more peripheral part of the cytoplasm of the cells. In contrast, after the 45-min incubation, the fluorescence was concentrated within small spots clustered in the center of the cells, close to the nucleus. These results are similar to the previous reports demonstrating that internalization of NTS proceeded through small endosome-like organelles and the internalized ligand accumulates to the core of the cell surrounding the nucleus (Austin et al. (1995) J. Mol. Endocrinol. 14: 157-69; Faure et al. (1995) J. Neurosci. 15: 4140-7).

### (6) Functional Receptor-Dimerization of GHSR1b and NTSR1

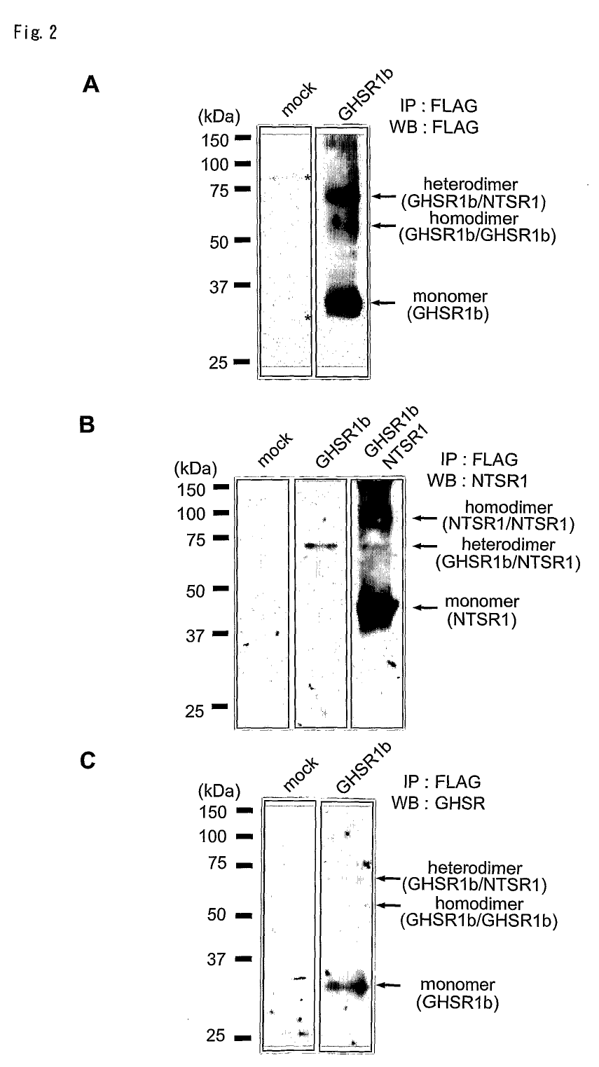

To examine direct association between GHSR1b and NTSR1, either or both of FLAG-tagged GHSR1b and FLAG-tagged NTSR1 were transiently expressed in COS-7 cells (FIG. 2A depicts the data of transient GHSR1b expression, and FIG. 2B the expression of both receptors). The COS-7 cells were confirmed by semiquantitative RT-PCR analysis to endogenously express both GHSR1b and NTSR1, but not NMU. Cell lysates pre-incubated with the cross-linking reagent were immunoprecipitated by anti-FLAQ anti-NTSR1, or anti-GHSR antibody. Co-precipitation of the following proteins was detected: GHSR1b monomer (˜30 kDa), NTSR1 monomer (˜45 kDa), GHSR1b/NTSR1 heterodimer (70-75 kDa), GHSR1b homodimer (˜60-65 kDa), and NTSR1 homodimer (˜90-95 kDa) (FIG. 2). No such species were detected when empty vector (mock) was transfected to COS-7 cells as the negative control. In the cells expressing only FLAG-tagged NTSR1 and those co-expressing both the FLAG-tagged receptors (NTSR1 and GHSR1b), similar results were observed. These results confirm an interaction between GHSR1b and NTSR1, implying the existence of GHSR1b/NTSR1 heterodimer.

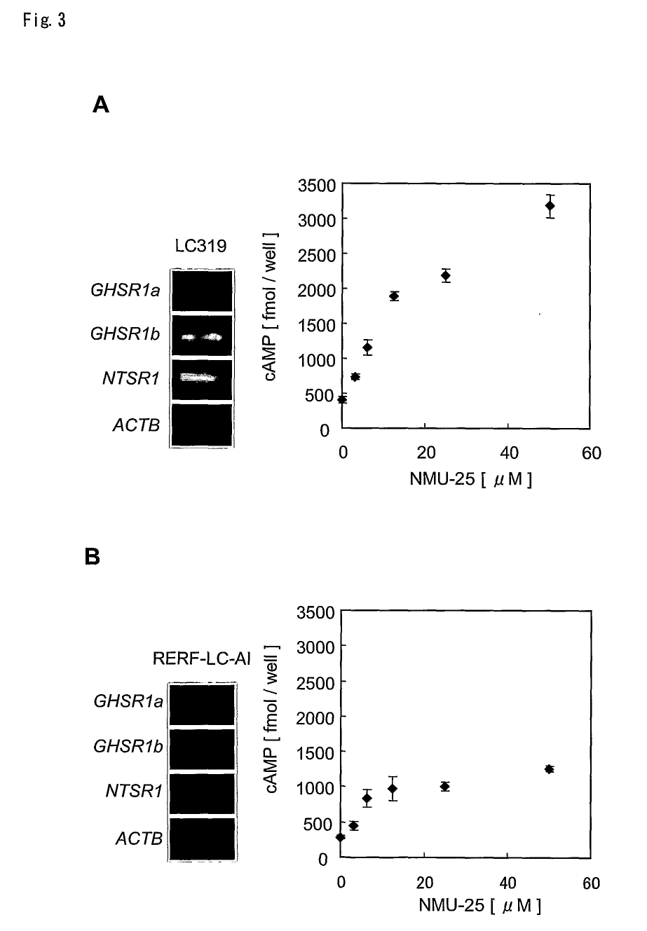

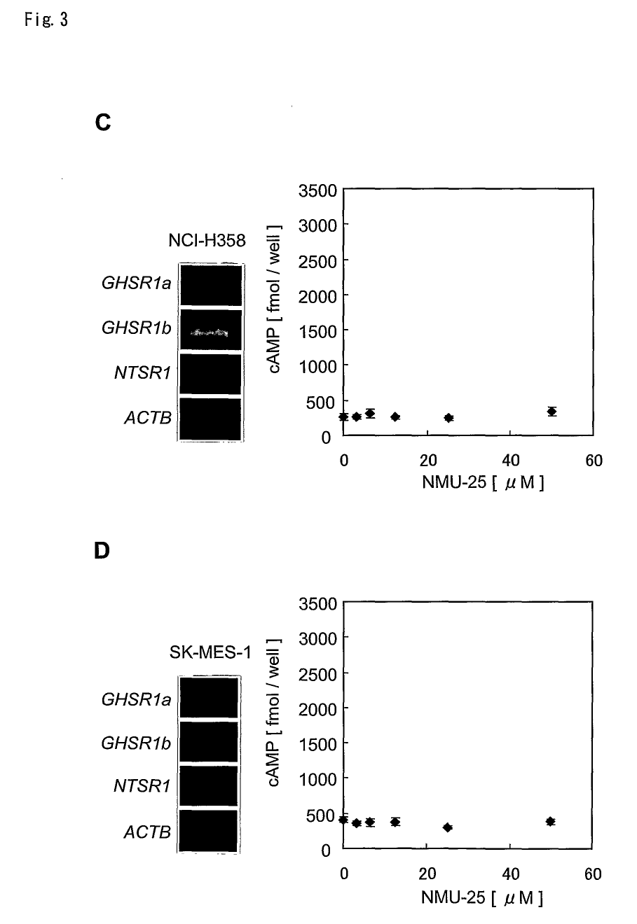

To further confirm the functional importance of the activation and heterodimerization of GHSR1b and NTSR1 at the signal transduction level, dose-dependent intracellular cAMP production by NMU-25 was examined in lung-cancer cell lines representing various expression patterns of the two receptors as detected by semiquantitative RT-PCR analysis (FIGS. 3A to 3D). In LC319 cells expressing high levels of both receptors, treatment with NMU-25 resulted in a marked and reproducible cAMP accumulation (FIG. 3A). RERF-LC-AI cells expressing both receptors at low levels showed not significant but low cAMP production in response to NMU-25 stimulation (FIG. 3B). NCI-H358 and SK-MES-1 cells expressing either of the receptors did not show detectable cAMP production (FIGS. 3C and 3D).

### (7) Identification of Downstream Genes of NMU

To further elucidate the NMU-signaling pathway, siRNA against NMU (si-NMU) or LUC (control siRNA) were transfected into LC319 cells, which overexpress NMU, and genes that were down-regulated in cells transfected with si-NMU were screened using cDNA microarray containing 32,256 genes. Through this approach, 70 genes whose expression was significantly decreased in accordance with the NMU suppression were selected by performing self-organizing map (SOM) clustering analysis (Kohonen (1990) Proceedings of the IEEE 78: 1464-80). Semiquantitative RT-PCR analysis confirmed reduction of candidate transcripts in a time-dependent manner in LC319 cells transfected with si-NMU, but not with control siRNA for LUC. The transactivation of these genes in response to the introduction of NMU expression in lung-cancer cell lines was also evaluated to finally identify six candidate NMU-target genes, FOXM1, GCDH, CDK5RAP1, LOC134145, NUP188, and one unannotated transcript (clone IMAGE: 3839141).

Among these selected six genes, FOXM1 mRNA level was found to be significantly elevated in clinical lung cancer cases and showed good concordance with the expression patterns of NMU and the two receptors, NTSR1 and GHSR1b. Hence, FOXM1 was focused for further analysis. To validate the induction of FOXM1 expression by NMU ligand-receptor signaling, LC319 cells expressing NTSR1 and GHSR1b were cultured under the presence of NMU-25 or BSA (control) at a final concentration of 25 μM in the culture media to confirm enhanced expression of FOXM1 in the NMU-treated cells. Then, the biological significance of FOXM1 activation in the NMU-signaling pathway was examined for the growth and survival of lung-cancer cells, using plasmids designed to express siRNA against FOXM1 (si-FOXM1). The transfection of si-FOXM1 into A549 or LC319 cells suppressed the expression of endogenous FOXM1 compared to the cells containing any of the three control siRNAs. In accordance with the reduced expression of FOXM1, A549 and LC319 cells showed significant decrease in cell viability and numbers of colonies.

Since FOXM1 was reported to function as a transcription factor and activates the expression of several cyclins (Wang et al. (2001) Proc. Natl. Acad. Sci. USA 98: 11468-73; Wang et al. (2002) Proc. Natl. Acad. Sci. USA 99: 16881-6), the expression of cyclin(s) in lung-cancer LC3 19 cells transfected with si-NMU was examined to detect the reduction of cyclin B1 (CCNB1) and cyclin A2 (CCNA2), but not cyclin D1 (CCND1) in a time-dependent manner after the transfection. Moreover, by semiquantitative RT-PCR analysis, good correlation of gene expression between FOXM1 and the two cyclins (CCNB1 and CCNA2) were detected in the clinical lung-cancer samples, which independently supports the hypothesis that NMU/FOXM1 transactivates CCNB1 and CCNA2 in lung-cancer cells. These results demonstrate that NMU, through the interaction with GHSR1b/NTSR1 heterodimer and subsequent activation of its downstream targets, such as FOXM1, could significantly affect the growth of lung-cancer cells.

### INDUSTRIAL APPLICABILITY

According to the present invention, a significant association of NMU expression with poor prognosis of NSCLC patients has been revealed. Based on this discovery, the present invention provides a method of and kit for assessing or determining the prognosis of lung cancer, in particular, NSCLC, by detecting the expression level of the NMU gene in a patient-derived biological sample. The method enables assessment of the prognosis of NSCLC using only routine procedures for tissue-sampling.

Furthermore, the present invention relates to a method for identifying or screening a therapeutic or preventive agent for cancer, in particular, lung cancer, by detecting compounds that inhibit the binding of the NMU protein with the heterodimer of GHSR1b and NTSR1 (GPCR heterodimer). According to the present invention, it was shown that NMU and the newly revealed GPCR heterodimer, the functional receptor of NMU, are not only overexpressed in the great majority of lung cancers, but are also essential for an autocrine growth-promoting pathway that activates various downstream genes including FOXM1. Thus, the present screening method might hold promise for development of a new therapeutic strategy for the treatment and prevention of lung cancer.

The data reported herein add to a comprehensive understanding of NSCLC, facilitate development of novel diagnostic strategies and provide clues for identification of molecular targets for therapeutic drugs and preventive agents. Such information contributes to a more profound understanding of carcinogenesis, and provides indicators for developing novel strategies for diagnosis, treatment and ultimately prevention of NSCLC.

All publications, databases, sequences, patents, and patent applications cited herein are herby incorporated by reference.

While the invention has been described in detail and with reference to specific embodiments thereof, it will be apparent to those skilled in the art that various changes and modifications can be made therein without departing from the spirit and scope of the invention, the metes and bounds of which are set by the appended claims.

1. A method for assessing the prognosis of a patient with lung cancer, which method comprises the steps of:
(1) detecting the expression level of the neuromedin U (NMU) gene in a biological sample collected from the patient;
(2) comparing the detected expression level to a control level; and
(3) determining the prognosis of the patient based on the comparison of (2).

2. The method of claim 1, wherein the lung cancer is non-small cell lung cancer (NSCLC).

3. The method of claim 1, wherein the control level is a good prognosis control level and an increase of the expression level compared to the control level is determined as poor prognosis.

4. The method of claim 3, wherein the increase is at least 10% greater than said control level.

5. The method of claim 1, wherein said method comprises determining the expression level of other lung cancer-associated genes.

6. The method of claim 1, wherein said expression level is determined by any one method selected from the group consisting of:
(a) detecting mRNA of the NMU gene;
(b) detecting the NMU protein; and
(c) detecting the biological activity of the NMU protein.

7. The method of claim 1, wherein said expression level is determined by detecting hybridization of a probe to a gene transcript of the NMU gene.

8. The method of claim 7, wherein the hybridization step is carried out on a DNA array.

9. The method of claim 1, wherein said expression level is determined by detecting the binding of an antibody against the NMU protein as the expression level of the NMU gene.

10. The method of claim 1, wherein said biological sample comprises sputum or blood.

11. A kit for assessing the prognosis of a patient with lung cancer, which comprises a reagent selected from the group consisting of:
(a) a reagent for detecting mRNA of the MU gene;
(b) a reagent for detecting the NMU protein; and
(c) a reagent for detecting the biological activity of the NMU protein.

12. The kit of claim 11, wherein the reagent is an antibody against the NMU protein.

13. A method of identifying a compound that inhibits the signal transduction by the NMU protein and the heterodimer consisting of growth hormone secretagogue receptor 1b (GHSR1b) and neurotensin receptor 1 (NTSR1), said method comprising the steps of:
(1) contacting a heterodimer of GHSR1b and NTSR1, or a functional equivalent thereof with the NMU protein in the existence of a test compound;
(2) detecting the signal transduction by the heterodimer and the NMU protein; and
(3) selecting the test compound that inhibits the signal transduction by the heterodimer and the NMU protein.

14. The method of claim 13, wherein the heterodimer is expressed on the surface of a living cell.

15. The method of claim 14, wherein the signal transduction by the heterodimer and the NMU protein is detected by any one method selected from the group consisting of:
(a) detecting the concentration of cAMP in the cell;
(b) detecting the activation of adenylate cyclase;
(c) detecting the activation of protein kinase A (PKA);
(d) detecting the expression of NMU target genes including FOXM1, GCDH, CDK5RAP1, LOC134145, and NUP188;
(e) detecting the change in subcellular localization of the heterodimer including ligand-induced internalization;
(f) detecting cell proliferation, transformation, or any other oncogenic phenotype of the cell; and
(g) detecting apoptosis of the cell.

16. A method of screening for a candidate compound for treating or preventing NSCLC, which comprises the steps of:
(1) identifying a compound that inhibits the signal transduction by the NMU protein and the heterodimer consisting of GHSR1b and NTSR1 through the method of claim 13; and
(2) determining the identified compound in step (1) as the candidate compound for treating or preventing NSCLC.

17. The method of claim 16, wherein the heterodimer is expressed on the surface of a living cell.

18. The method of claim 17, wherein the signal transduction by the heterodimer and the NMU protein is detected by any one method selected from the group consisting of:
(a) detecting the concentration of cAMP in the cell;
(b) detecting the activation of adenylate cyclase;
(c) detecting the activation of protein kinase A (PKA);
(d) detecting the expression of NMU target genes including FOXM1, GCDH, CDK5RAP1, LOC134145, and NUP188;
(e) detecting the change in subcellular localization of the heterodimer including ligand-induced internalization;
(f) detecting cell proliferation, transformation, or any other oncogenic phenotype of the cell; and
(g) detecting apoptosis of the cell.
